(19) United States
(12) Patent Application Publication
(10) Pub. No.: US 2005/0203191 A1
(43) Pub. Date: Sep. 15, 2005

(54) 1-Aminocyclohexane derivatives for the treatment of agitation and other behavioral disorders, especially those associated with alzheimer's disease

(75) Inventors: Scott McDonald, Chatham, NJ (US); Barry Reisberg, New York, NY (US); Steven Ferris, New York, NY (US); Hans-Joerg Moebius, Frankfurt (DE); Albrecht Stoffler, Frankfurt Am Main (DE)

Correspondence Address: DARBY & DARBY P.C., P. O. BOX 5257, NEW YORK, NY, 10150-5257, US

(73) Assignee: Forest Laboratories, Inc., New York, NY (US)

(21) Appl. No.: 11/074,326

(22) Filed: Mar. 3, 2005

Related U.S. Application Data
(60) Provisional application No. 60/550,171, filed on Mar. 3, 2004.

(51) Int. Cl. A61K 31/13; A61K 31/445

(52) U.S. Cl. 514/662; 514/319

(57) ABSTRACT

The present invention relates to the treatment of behavioral disorders, especially agitation, associated with a central nervous system (CNS) disorder, especially Alzheimer's disease (AD), cerebrovascular disease (VaD), or Down's Syndrome, in a mammal, comprising administering to said mammal an 1-aminocyclohexane, alone or in combination with a acetylcholinesterase inhibitor. In one embodiment, the 1-aminocyclohexane is memantine.

This application claims priority from U.S. Provisional Application Ser. No. 60/550,171, filed on Mar. 3, 2004, the disclosure of which is incorporated herein in its entirety.

### FIELD OF THE INVENTION

The present invention relates to the treatment of behavioral disorders associated with a central nervous system (CNS) disorder, especially Alzheimer's disease (AD), cerebrovascular disease (VaD), or Down's Syndrome, in a mammal, comprising administering to said mammal an 1-aminocyclohexane, alone or in combination with a acetylcholinesterase inhibitor.

### BACKGROUND OF THE INVENTION

Alzheimer's Disease

Dementia is a serious disorder affecting as many as 10% of individuals older than 65 years and more than 24% of those older than 85 years (Hofman et al., Int. J. Epidemiol., 1991, 20:736-748; Jorm and Jolley, Neurology, 1998, 51:728-733; Lobo et al., Neurology, 2000, 54(Suppl. 5):S4-S9). Alzheimer's disease (AD) is an increasingly prevalent form of neurodegeneration that accounts for approximately 50% -60% of the overall cases of dementia among people over 65 years of age. AD is characterized clinically by progressive loss of memory, cognition, reasoning, judgement, and emotional stability that gradually leads to profound mental deterioration and ultimately death. AD is a progressive disorder with a mean duration of around 8.5 years between onset of clinical symptoms and death. AD is believed to represent the fourth most common medical cause of death and affects about 4-5 million people in the United States. Prevalence of AD doubles every 5 years beyond age 65 (National Institute on Aging: Prevalence and costs of Alzheimer's disease. Progress Report on Alzheimer's Disease. NIH Publication No. 99 3616, November 1998; Polvikoski et al., Neurology, 2001, 56:1690-1696). AD currently affects about 15 million people world-wide (including all races and ethnic groups) and owing to the relative increase of elderly people in the population, its prevalence is likely to increase over the next two to three decades. AD is at present incurable. No treatment that effectively prevents AD or completely reverses its symptoms and course is currently known.

AD is associated with death of pyramidal neurons and loss of neuronal synapses in brains regions associated with higher mental functions (Francis et al., 1999, J. Neurol. Neurosurg. Psychiatry, 66:137-147). The brains of individuals with AD exhibit characteristic lesions termed senile (or amyloid) plaques, amyloid angiopathy (amyloid deposits in blood vessels) and neurofibrillary tangles. Approved treatments for AD include acetylcholinesterase inhibitors or NMDA receptor antagonists.

Acetylcholinesterase Inhibitors

AD is associated with a profound loss of cholinergic neurons within the nucleus basalis of Meynert (Perry et al., Br. Med. J., 1978, 2:1456-1459; Geula and Mesulam, Cholinergic systems and related neuropathological predilection patterns in Alzheimer disease. In: Alzheimer's Disease. Terry et al. eds.; New York: Raven Press; 1994, pp. 263-291). The signaling in these neurons is mediated by the extracellularly released neurotransmitter acetylcholine (ACh). Recognition of the role of dysfunction of ACh signaling system in the cognitive impairments associated with AD as well as a number of other neurological and psychiatric disorders, including Parkinson's disease, schizophrenia, epilepsy, depression, obsessive compulsive disorders, and bipolar disorders, has led to the development of drugs that selectively enhance cholinergic function by inhibition of the cholinergic catabolic enzyme acetylcholinesterase (AChE). AChE destroys ACh after the latter has been secreted into the synaptic clefts (Goff and Coyle, Am. J. Psychiatry, 2001, 158: 1367-1377).

At present, the most widely clinically used acetylcholinesterase inhibitors (AChEI) are tacrine (THA; 1,2,3,4-tetrahydro-9-aminoacridine hydrochloride), DFP (diisopropylfluorophosphate), physostigmine, donepezil, galantamine, and rivastigmine. Many of AChEI selectively inhibit AChE, but agents that also target butyrylcholinesterase (BuChE) may provide added benefits as AD progresses and ACh regulation may become increasingly dependent on BuChE. Dual inhibition may also help to slow the formation of amyloidogenic compounds (Ballard, Eur. Neurol., 2002, 47:64-70).

Donepezil ([(R,S)-1-benzyl-4-[(5,6-dimethoxy-1-indanon)-2yl]-methylpiperidine hydrochloride]; ARICEPT, previously E-2020) is a reversible, noncompetitive, piperidine-type AChEI, which is selective for AChE rather than BuChE (Sugimoto et al., Curr. Med. Chem., 2000, 7:303-39). Dooley et al. (Drugs Aging, 2000, 16:199-226) have demonstrated that donepezil administered at doses of 5 and 10 mg/day significantly improved cognition and global clinical function compared with placebo in short term trials (14 to 30 weeks) in 161 to 818 patients with mild to moderate AD (see also Rogers et al., Arch. Int. Med., 1998; 158:1021-1031). Long-term efficacy data obtained in these studies suggest that improvements in cognition, global function or activities of daily living (ADL) are maintained for about 21 to 81 weeks

Galantamine (REMINYL) is a reversible, competitive, tertiary alkaloid AChEI, which is selective for AChE rather than BuChE. As demonstrated by Scott et al. (Drugs, 2000; 60:1095-122), 285 to 978 patients with mild to moderate AD receiving galantamine at doses 16 or 24 mg/day achieved significant improvements in cognitive and global symptoms relative to placebo recipients in trials of 3 to 6 months' duration.

Rivastigmine (EXELON) is a dual inhibitor of AChE and BuChE that has demonstrated benefits across the spectrum of AD severity (Ballard, Eur. Neurol., 2002, 47:64-70). Unlike tacrine and donepezil, which are classified as short-acting or reversible agents, rivastigmine is an intermediate-acting or pseudo-irreversible agent, which inhibits AChE for up to 10 hours. Preclinical biochemical studies indicated that rivastigmine has central nervous system (CNS) selectivity over peripheral inhibition. Rivastigmine was shown to ameliorate memory impairment in rats with forebrain lesions; and in the two large multicenter clinical trials (total 1324 patients) at doses 6-12 mg/day it was superior to placebo on three cognitive and functioning scales (Jann, Pharmacotherapy, 2000, 20:1-12).

NMDA Receptor Antagonists

The excessive or pathological activation of glutamate receptors, particularly those that are selectively activated by N-methyl-D-aspartate (NMDA), has also been implicated in the processes that underlie the degeneration of cholinergic cells in the brains of AD patients (Greenamyre et al., Neurobiol. Aging, 1989, 10:593-602; Francis et al., J. Neurochem., 1993, 60:263-291; Li et al., J. Neuropathol. Exp. Neurol., 1997, 56:901-911; Wu and Rowan, Neuroreport, 1995, 6:2409-2413). The NMDA receptor is very well established to be pivotal for several physiologic synaptic plasticity processes, e.g., memory and learning (Collinridge and Singer, Trends Pharmacol. Sci., 1990, 11: 290-296). The functioning of the NMDA receptor requires the activation of both the agonist binding site for glutamate and the allosteric co-agonist site which is activated by glycine and D-serine (Kleckner and Dingledine, Science, 1988, 241:835-837; McBain et al., Mol. Pharmacol., 1989, 36:556-565; Danysz and Parsons, Pharmacol. Rev., 1998, 50:597-664). Activation of the D-serine-sensitive modulatory site on the NMDA receptor has been shown to be a prerequisite for induction of long-term potentiation (Bashir et al., Neurosci Lett., 1990, 108:261-266), an in vitro correlate of memory and learning. Further, the cognitive deficits associated with psychiatric disorders such as schizophrenia have been shown to be alleviated by oral treatment with D-serine (Tsai et al., Biol Psychiatry, 1998, 44:1081-1089).

Even though NMDA receptor activation is critical for learning, moderate affinity uncompetitive NMDA receptor antagonists have been found to correct/reverse cognitive impairment in both human AD and animal models of Alzheimer's dementia. To the degree that excessive glutamatergic function is a contributor in AD, effective pharmacological antagonism of the NMDA receptor, particularly by open channel blockers, may be able to slow the progression of AD (Parsons et al., Neuropharmacol., 1999, 38:735-767; Danysz and Möbius, 2002, Alzheimer's Disease Neuroprotection—Therapeutic Potential of Ionotropic Glutamate Receptor Antagonists and Modulators, In: Therapeutic Potential of Ionotropic Glutamate Receptor Antagonists and Modulators, Lodge et al. eds., 2002, in press, F. P. Graham Publishing Co., New York).

NMDA receptor antagonists potentially have a wide range of therapeutic applications in numerous CNS disorders such as acute neurodegeneration (e.g., associated with stroke and trauma), chronic neurodegeneration (e.g., associated with Parkinson's disease, AD, Huntington's disease, and amyotrophic lateral sclerosis [ALS]), epilepsy, drug dependence, depression, anxiety, and chronic pain (for reviews see: Parsons et al., Drug News Perspect., 1998, 11:523-533; Parsons et al., 1999, supra; Jentsch and Roth, Neuropsychopharmacology, 1999, 20: 201-205; Doble, Therapie, 1995, 50: 319-337). Functional inhibition of NMDA receptors can be achieved through actions at different recognition sites within the NMDA receptor complex, such as: the primary transmitter site (competitive), the phencyclidine site located inside the cation channel (uncompetitive), the polyamine modulatory site and the strychnine-insensitive, co-agonistic glycine site (glycine B) (Parsons et al., 1999, supra).

NMDA receptor inhibitors are likely to impair normal synaptic transmission and thereby cause numerous side effects. Indeed, many NMDA receptor antagonists identified to date produce highly undesirable side effects at doses within their putative therapeutic range. Supporting this, clinical trials failed to support good therapeutic utility due to numerous side effects for such NMDA receptor antagonists as Dizocilpine ((+)MK-801; (+)-5-methyl-10,11-dihydro-5H-dibenzocyclohepten-5,10-imine maleate), Cerestat (CNS-1102), Licostinel (ACEA 1021), Selfotel (CGS-19755), and D-CPP-ene (Leppik, Epilepsia, 1998, 39 (Suppl 5):2-6; Sveinbjornsdottir et al., Epilepsia, 1993, 34:493-521; SCRIP 2229/30, 1997, p. 21). The challenge in the field has therefore been to develop NMDA receptor antagonists that prevent the pathological activation of NMDA receptors but allow their physiological activity.

Memantine and Neramexane (1-amino-3,5-dimethyl adamantine, and pharmaceutically acceptable salts thereof) is an analog of 1-amino-cyclohexane (disclosed, e.g., in U.S. Pat. Nos. 4,122,193; 4,273,774; 5,061,703). Neramexane (1-amino-1,3,3,5,5-pentamethylcyclohexane) is also a derivative of 1-aminocyclohexane (disclosed, e.g., in U.S. Pat. No. 6,034,134). Memantine, neramexane as well as some other 1-aminoalkyl-cyclohexanes are systemically-active noncompetitive NMDA receptor antagonists having moderate affinity for the receptor. They exhibit strong voltage dependent characteristics and fast blocking/unblocking kinetics (Parsons et al., 1999, supra; Görtelmeyer et al., Arzneim-Forsch/Drug Res., 1992, 42:904-913; Winblad et al., Int. J. Geriat. Psychiatry, 1999, 14:135-146; Rogawski, Amino Acids, 2000, 19: 133-49; Danysz et al., Curr. Pharm. Des., 2002, 8:835-43; Jirgensons et. al., Eur. J. Med. Chem., 2000, 35: 555-565). These compounds dissociate from the NMDA receptor channels much more rapidly than the high affinity NMDA receptor antagonists such as (+)MK-801 and attenuate disruption of neuronal plasticity produced by tonic overstimulation of NMDA receptors probably by causing an increase of the signal-to-noise ratio. Due to their relatively low affinity for the receptor, strong voltage dependency and fast receptor unblocking kinetics, these compounds are essentially devoid of the side effects of other NMDA receptor antagonists at doses within the therapeutic range (Kornhuber et al., Eur. J. Pharmacol., 1991, 206:297-311). Indeed, memantine has been applied clinically for over 20 years showing good tolerability with approximately 700,000 patient-years exposure.

Memantine, neramexane as well as other 1-aminoalkylcyclohexanes have been suggested to be useful in alleviation of various progressive neurodegenerative disorders such as dementia in AD, Parkinson's disease, and spasticity (see, e.g., U. S. Pat. Nos. 5,061,703; 5,614,560, and 6,034,134; Parsons et al., 1999, supra; Möbius, ADAD, 1999, 13:S172-178; Danysz et al., Neurotox. Res., 2000, 2:85-97; Winblad and Poritis, Int. J. Geriatr. Psychiatry, 1999, 14:135-146; Görtelmeyer et al., 1992, supra; Danysz et al., Curr. Pharm. Des., 2002, 8:835-843; Jirgensons et. al., Eur. J. Med. Chem., 2000, 35: 555-565). These diseases result from disturbances of glutamatergic transmission, i.e., the excessive influx of calcium through NMDA receptor channels, leading to the destruction of brain cells in specific brain areas (Choi, J. Neurobiol., 23: 1261-1276, 1992; Rothman and Olney, Trends Neurosci., 10: 299, 1987; Kemp et al., Trends Pharmacol. Sci., 8: 414, 1987). Chronic treatment of adult rats with memantine has been shown to enhance the formation of hippocampal long-term potentiation, increase the durability of synaptic plasticity, improve spatial memory abilities, and reverse the memory impairment produced by NMDA receptor agonists (Barnes et al., Eur. J. Neurosci., 1996; 8:65-571; Zajaczkowski et al., Neuropharm, 1997, 36:961-971).

1-Aminocyclohexane derivatives, and specifically memantine, have also been suggested to be useful in the treatment of AIDS dementia (U.S. Pat. No. 5,506,231), neuropathic pain (U.S. Pat. No. 5,334,618), cerebral ischemia (U.S. Pat. No. 5,061,703), epilepsy, glaucoma, hepatic encephalopathy, multiple sclerosis, stroke, and tardive dyskinesia (Parsons et al., 1999, supra Relatively high doses of memantine and neramexane also were shown to selectively block thermal hyperalgesia and mechanical allodynia in some models of chronic and neuropathic pain without obvious effects on motor reflexes. 1-Aminoacyclohexane derivatives were also demonstrated to possess immunomodulatory, antimalarial, anti-Borna virus, and anti-Hepatitis C activities (see, e.g., U.S. Pat. No. 6,034,134 and references cited therein).

1-Aminocyclohexane derivatives such as memantine and neramexane have also been suggested to function via non-NMDA-mediated pathways (see U.S. patent application Ser. No. 09/597,102 and its corresponding international patent application PCT EP 01/06964 published as WO 01/98253 on Dec. 27, 2001; U.S. Pat. No. 6,034,134). Memantine was shown to inhibit 5HT3-mediated current (in the native N1E-115 and heterologous HEK-293 cells) and NMDA receptor-mediated currents (in rat hippocampal slices) with approximately equal affinity (Parsons et al., 1999, supra; Rammes et al., 2001, Neurosci. Lett., 306:81-84). 5HT3 receptor antagonists are known to improve learning and memory in animals (Carli et al., 1997, Behav. Brain Res., 82:185-194; Reznik and Staubli, 1997, J. Neurophysiol., 77:517-521).

As disclosed above, the loss of cholinergic neurons within the basal forebrain, which underlies various aspects of dementia, may result from the disruption in ACh-mediated signalling and/or excessive activation of NMDA receptors. Accumulating experimental evidence indicates that ACh and NMDA receptor-mediated signalling systems are interconnected, i.e., that blockade of NMDA receptors can increase the extracellular release of ACh. It has been demonstrated that systemic administration of the NMDA receptor antagonist, (+)MK-801, produces a dose-dependent increase in the extracellular release of ACh in rat parietal and frontal cortices (Hasegawa et al., 1993, Neurosci. Lett., 150:53-56; Aquas et al., 1998, Neuroscience, 85:73-83). Similarly, intracerebroventricular (i.c.v.) administration of another NMDA receptor antagonist, CPP, has been shown to increase ACh release in the rat parietal cortex and hippocampus (Giovannini et al., 1994, Neurochem. Intl., 25:23-26; Giovannini et al., 1994, J. Neurosci., 14:1358-1365). It has been proposed that glutamate, by acting through the NMDA receptors on GABAergic and noradrenergic neurons, maintains a tonic inhibitory control over the basal forebrain cholinergic neurons projecting to the cerebral cortex (Kim et al., 1999, Mol. Psychiat., 4:344-352). Based on this circuit, in addition to possible blocking of NMDA overactivation, systemic administration of an NMDA receptor antagonist would be expected to decrease the inhibitory control of GABA on ACh neurons resulting in the increased release of ACh in the cortex.

Agitation

Agitation is an umbrella term that can refer to a range of behavioral disturbances or disorders, including aggression, combativeness, hyperactivity, and disinhibition. Agitation is a nonspecific constellation of relatively unrelated behaviors that can be seen in a number of different clinical conditions, usually presenting a fluctuating course. Agitation may be caused by a number of different medical conditions and drug interactions or by any circumstances that worsen the person's ability to think.

Multiple underlying pathophysiologic abnormalities are mediated by dysregulations of dopaminergic, serotonergic, noradrenergic, and GABAergic systems. Agitation is characterized by non-productive, diffuse and excessive overactivity-both motor (akathisia) and cognitive, and accompanied by an inner unpleasant tension.

Common medications used to treat agitation include beta blockers such as propranolol and pindolol, anxiety medications such as buspirone, anti-convulsants such as valproate and lamotrigine, anti-psychotics such as haloperidol and other high-potency dopamine-blocking agents and atypical anti psychotics.

Agitation and dementia. Agitation in elderly patients with dementia, such as senile dementia associated with Alzheimer's Disease (SDAT) and vascular dementia, contributes to additional stress for caregivers and often requires additional treatment with medication. Specific subtypes of agitation include that associated with delirium, psychosis, depression (with or without psychosis), anxiety, insomnia, sundowning (progression of agitation in the evening hours), aggression and anger, and pain (e.g., osteoarthritic). The foregoing subtypes of agitation can be caused from conditions such as urinary tract infections, poor nutrition, respiratory infection, recent stroke, occult head trauma, e.g., from a recent fall, pain, constipation, congestive heart failure, orthostatic hypotension, chronic obstructive pulmonary disease, hypothyroidism, diabetes, alcohol or other substance abuse, substance abuse-withdrawal, long bone fracture, e.g., from a recent fall. Agitation can also be caused by medication or other substances used to treat the underlying syndrome associated with agitation.

A panel of expert consensus guidelines suggests the following drug therapy as first line treatment for the above-referenced sub-types of agitation:
Delirium-conventional high potency antipsychotics, e.g., haloperidol
Psychosis-risperidone and conventional high potency antipsychotics, olanzapine, divalproex and trazodone
Depression-antidepressants
Anxiety-buspirone, trazodone and SSRIs
Insomnia-trazodone and benzodiazepines for acute management
Sundowning-trazodone, risperidone, olanzapine, and conventional high potency antipsychotics
Aggression and anger-trazodone, divalproex, SSRIs, and buspirone
Pain-tricyclic antidepressants, SSRIs, and trazodone

(Treatment of Agitation in Dementia, A Postgraduate Medicine Special Report, March 1998; Eds. Alexopoulos et al., The McGraw-Hill Companies, Inc.)

Agitation and depression. Frequently, patients with severe depression may develop agitation that cannot be controlled by drug treatment such as benzodiazepines. Benazzi has also characterized a sub-population of depressed patients who exhibit racing thoughts and psychomotor agitation (Psychiatry Res. 2003;120(3):273-82).

Agitation associated with SSRI withdrawal. When SSRI antidepressants are abruptly discontinued, withdrawal symptoms such as anxiety, agitation, sleep disturbances, movement disorders, restlessness and delirium often present (Rosenbaum et al., J. Clin. Psychiatry 1997; 58(suppl.7): 37-40).

Agitation in pediatric populations. In addition to agitation associated with dementia, agitation is also common in syndromes affecting children such as depression, attention deficit disorder (with and without hyperactivity), conduct disorder, oppositional defiant disorder, and separation anxiety disorder. There is also evidence that the pediatric population becomes agitated following anesthesia, especially sevofluorane (Voepel-Lewis et al., Anesth Analg. 2003;96(6):1625-30).

Agitation associated with mood disorders. Agitation is frequently associated with mood disorders such as bipolar disorder and schizophrenia. In bipolar disorder, agitation usually presents during acute manic states, but can also present during mixed depressive states. Agitation is also associated with schizophrenia, and acute forms of agitation are typically treated with intramuscular ziprasidone and olanzapine, but it has recently been shown that oral olanzapine, when administered in a rapid initial dose escalation (40 mg on days 1 and 2, 30 mg on days 3 and 4, 5-20 mg thereafter) exhibited superior improvement (J. Clin. Psychopharmacol. 2003; 23: 342-348).

Post-operative agitation. The average incidence of post-operative agitation is about 11-40% and can result in increase in the incidence of major complications, increases in admission to rehabilitation centers, increased duration of hospital stays, and is predictive of mortality. Agitation following cardiac surgery, such as coronary artery bypass graft (CABG) surgery, is especially common. Post-operative agitation can be caused by factors such as hypoxemia, hypotension, metabolic disorders, residual effects of anesthetics, sepsis, or cerebral embolism.

ICU agitation. Agitation is a commonly encountered problem in the ICU. Agitated patients have the potential to jeopardize their own care by disconnecting various life-sustaining modalities. Additionally, these patients pose a risk to the nurse and physician care providers and compromise the care of other ICU patients by monopolizing limited provider care time. In a recent study, nurses and physicians described agitated behavior in 71% of patients occurring during 58% of total patient days; the behavior was severe or dangerous in 46% of patients during 30% of total patient days. (Fraser et al. Pharmacotherapy 2000; 20:75-82). Pain and anxiety are typical causes of ICU agitation.

Agitation due to substance-abuse withdrawal. Agitation, especially manifesting as psychomotor agitation, is a symptom of alcohol and drug (including narcotics) withdrawal. For alcohol withdrawal that cannot be managed with supportive care, benzodiazepines, especially diazepam and chlordiazepoxide, are the drugs of choice. Barbiturates, beta-blockers, and antipsychotics are generally not recommended as first-line therapy. Several drugs in other classes, including carbamazepine and clonidine, have been shown to be about as effective as benzodiazepines in a few studies, but the studies were small, the patients were usually in mild withdrawal, and validated instruments for assessing withdrawal were often not used. Some agents, such as beta-blockers, may play a role as adjuncts to, not replacements for, benzodiazepine therapy.

Psychomotor agitation as a symptom is also associated with withdrawal from cocaine, nicotine, naltrexone-associated detoxification (Armstrong et al., Acad Emerg Med. 2003;10(8):860-6), opioids (Puntillo et al., Heart Lung. 1997;26(4):317-24), benzodiazepines, gabapentin (Norton et al., Clin Neuropharmacol. 2001;24(4):245-6) and gamma-hydroxybutyrate (Craig et al., J Emerg Med. 2000;18(1):65-70).

Agitation resulting from traumatic brain injury. Traumatically brain-injured (TBI) patients compose a large portion of the population treated in rehabilitation programs. There has been an increase in the number and acuity of agitated clients in this population (Herbel et al., Rehabil Nurs. 1990;15(2):66-9). Pharmacological management of TBI includes beta-blockers, anti-convulsants, dopaminergic drugs, and anti-psychotics (Fleminger et al., Cochrane Database Syst Rev. 2003;(1):CD003299).

Agitation in terminally ill patients. Communication capacity impairment and agitated delirium are frequently observed in terminally ill cancer patients, and are significantly correlated with a higher dose requirement of opioids and the presence of icterus (Morita et al., J Pain Symptom Manage. 2003; 26(3):827-34).

The present inventors have conceived and demonstrated for the first time that the clinical administration of an 1-aminocyclohexane derivative such as memantine or neramexane, alone or in combination with an AChEI such as galantamine, tacrine, donepezil, or rivastigmine, is an unexpectedly valuable pharmacotherapeutic approach to the treatment of behavioral disorders associated with a central nervous system (CNS) disorder, especially Alzheimer's disease (AD), cerebrovascular disease (VaD), or Down's Syndrome. The present invention demonstrates that, when administered alone or in combination with AChEIs, to subjects with AD, the effects of 1-aminocyclohexane derivatives unexpectedly relieved associated behavioral symptoms such as agitation. This positive effect on behavioral symptoms was shown in the absence of co-therapy with sedatives such as antipsychotics.

Memantine is currently approved in Europe and the United States for the treatment of moderate-to-severe AD. Memantine has also unexpectedly proven useful for treatment of mild-to-moderate AD (see U.S. patent application Ser. No. 11/030,584, filed Jan. 5, 2005, incorporated by reference herein in its entirety). The present invention unexpectedly demonstrates that in patients with AD, memantine reduces agitation. This finding is expanded to other behavioral disorder for which agitation presents.

### SUMMARY OF THE INVENTION

The present invention relates to the treatment of behavioral disorders, particularly those associated with underlying conditions such as a central nervous system (CNS) disorder, especially Alzheimer's disease (AD), cerebrovascular disease (VaD), or Down's Syndrome, or with a traumatic brain injury, in a mammal comprising administering to said mammal an 1-aminocyclohexane, alone or in combination with a acetylcholinesterase inhibitor.

In one embodiment, the behavioral disorder includes, for example, delusions, hallucinations, agitation/aggression, depression/dysphoria, anxiety, elation/euphoria, apathy/indifference, disinhibition, irritability/lability, aberrant motor activity, nighttime behavior, and appetite/eating changes.

More specifically, the behavioral disorder treated according to the instant invention is agitation; in a particular embodiment the agitation is associated with depression;
in another embodiment the agitation is associated with withdrawal of a selective serotonin reuptake inhibitor;
in a further embodiment, the agitation is associated with a mood disorder, e.g., schizophrenia or bipolar disorder;
in yet another embodiment, the agitation is associated with substance abuse withdrawal;
in another embodiment, the agitation is associated with traumatic brain injury;
in a further embodiment, the agitation is associated with terminal illness;

The present invention also provides a method for treating agitation associated with a pediatric disorder;
in one embodiment, the pediatric disorder is depression, attention deficit disorder (with and without hyperactivity), conduct disorder, oppositional defiant disorder, or separation anxiety disorder.

The present invention also provides a method for treating agitation that presents in the intensive care unit, or that arises post-operatively, such as from anesthesia.

The present invention also provides a method for treating agitation associated with a CNS disorder or traumatic injury.

The present invention provides a method of treating agitation associated with Alzheimer's disease, including without limitation, moderate to severe Alzheimer's disease

In one embodiment, the agitation is severe, e.g., as determined by a patient having an NPI agitation score of greater than or equal to 4, including but not limited to those determined to be top 25% highest scoring population according to the NPI scale.

The present invention also provides a method of treating a behavioral disorder comprising administering to a patient an antipsychotic, in addition to memantine and other aminocyclohexane derivatives and their pharmaceutically acceptable salts.

Further aspects of the invention include pharmaceutical compositions comprising therapeutically effective amounts of 1-aminocyclohexanes, alone or in combination with acetylcholinesterase inhibitors and, optionally, at least one pharmaceutically acceptable carrier or excipient.

In one embodiment, the 1-aminocyclohexane is administered in an amount from 5-200 mg/kg per day.

In another embodiment, the 1-aminocyclohexane is administered in an amount from 5-200 mg/kg per day along with a acetylcholinesterase inhibitor at a does of 5-200 mg/kg per day.

In a further embodiment, the present invention provides pharmaceutical dosage form for treatment of behavioral disorders associated with a central nervous system (CNS) disorder, especially Alzheimer's disease (AD), cerebrovascular disease (VaD), or Down's Syndrome, comprising a therapeutically effective amount of an 1-aminocyclohexane according to the instant invention and, optionally, an acetylcholinesterase inhibitor and, optionally, a pharmaceutically acceptable carrier or excipient.

In a specific embodiment of the instant invention, the 1-aminocyclohexane is selected from memantine, neramexane, or derivatives thereof, with the terms including pharmaceutically acceptable salts of these active agents.

In yet a further specific embodiment of the instant invention, when an acetylcholinesterase inhibitors used, it is selected from tacrine (THA; 1,2,3,4-tetrahydro-9-aminoacridine hydrochloride), DFP (diisopropylfluorophosphate), physostigmine, donepezil, galantamine, and rivastigmine.

### DETAILED DESCRIPTION OF THE INVENTION

As specified above, in one aspect, the instant invention provides a novel method for treating, preventing, arresting, delaying the onset of and/or reducing the risk of developing, or reversing behavioral disorders associated with a central nervous system (CNS) disorder, especially Alzheimer's disease (AD), cerebrovascular disease (VaD), or Down's Syndrome, in a mammal comprising administering to said mammal an effective amount of a 1-aminocyclohexane, alone or in combination with an acetylcholinesterase inhibitor (AChEI).

The following neuopsychiatric scales were used to assess selected behavioral disorders associated with Alzheimer's diseases for treatment according to the methods of the present invention.

Cognitive, Functional, and Global Scales

The Alzheimer's Disease Assessment Scale-cognitive subscale, or ADAS-cog, comprises an 11-item scale that is used to assess the severity of selected areas of cognitive impairment (memory, language, orientation, reason and praxis). Scores range from 0 to 70 with lower scores indicating lesser severity and a score of 70 representing the worst cognitive impairment. Its use in assessing and following changes in patients with mild to moderate Alzheimer's disease has been extensively validated. The ADAS-cog is administered at each clinic visit starting with the Baseline visit.

The Clinician's Interview-Based Impression of Change including caregiver information, or CIBIC-Plus, is a global rating that was derived through an independent, comprehensive interview with the patient and caregiver by an experienced rater/clinician who was barred from knowledge of all other psychometric test scores (after Baseline visit) conducted as part of this protocol and who is not otherwise familiar with the patient (Reisberg et al., Alzheimer Dis. Assoc. Disord. 1997; 11(Suppl.3): 8-18). Scores 1-3 indicate improvement; Score 4 indicates no change (as compared to baseline); Scores 5-7 indicate worsening. The CIBIC-rater assesses disease severity at Baseline. Using the results from Baseline for reference, the rater then interviews the patient and caregiver at the end of e.g., Weeks 4, 8, 12, 18 and 24 (or upon early termination), to obtain an “Impression of Change” rating. The format for this scale was derived from the Alzheimer's Disease Cooperative Study—Clinician's Global Impression of Change scale (ADCS-CGIC) (Schneider, L. et al, 1997). The CIBIC-plus is administered at each clinic visit starting with the Baseline visit.

The Alzheimer's disease Cooperative Study-Activities of Daily Living, or ADCS-ADL, inventory consists of 23 questions used to measure the functional capabilities of patients with dementia (Galasko et al., Neurobiol. Aging 2000; 21(Suppl.1): 168). These questions are selected from a larger set of 49 questions in the original ADL scale. A more common selection is of 19 questions from the same 49 question group (ADCL-ADL19). Each ADL item comprises a series of hierarchical sub-questions, ranging from the highest level of independent performance of each ADL to complete loss. The ADSC-ADL Inventory total score ranges from 0 (lower functioning status) to 78 (higher functioning status). A higher score indicates a better functioning status. The inventory is performed by interviewing a person who is in close contact with the patient and covers the most usual and consistent performance of the patient over the preceding four weeks (Galasko et al., 1997). The ADCS-ADL is administered at each clinic visit starting with the Baseline visit.

The Severe Impairment Battery, or SIB, was developed to assess a range of cognitive functioning in individuals who are too impaired to complete standard neuro-psychological tests. The SIB focuses on the gap left by other instruments by providing an opportunity to gather direct performance-based data on a wide variety of low level tasks which take into account the specific behavioral and cognitive deficits associated with severe dementia. The SIB evaluates cognitive abilities at the lower end of the range. It is composed of very simple one-step commands which are presented in conjunction with gestural cues, and it allows for non-verbal and partially correct responses as well as for simpler response modes such as matching. The SIB is designed to be psychometrically reliable and allows for repeated assessments. Each subscale yields scores that are downward extensions of instruments used to assess mild to moderate dementia. The six major subscales are: attention; orientation; language; memory; visuo-spatial ability; construction. In addition, there are also brief evaluations of praxis, social interaction and orienting to name.

The Neuropsychiatric Inventory, or NPI, is a validated scale that assesses behavioral disturbances in patients with dementia (Cummings et al., 1994). It provides both a total score (sum of 12 domain scores) as well as scores for a number of subscales (delusions or paranoia; visual and auditory hallucinations; agitation or aggression; depressed mood or dysphoria; anxiety; elation or euphoria; apathy or indifference; impulsive disinhibition; irritability or lability (decreased coping); motor disturbance; nighttime behaviors; appetite or eating (e.g. weight loss). The NPI total score ranges from 0 (higher functioning status) to 144 (lower functioning status). For each subscale, both the frequency and the severity of each behavior is measured. Severity (1-mild to 3-severe); distress (0-no distress to 5-extremely distressing). The NPI is based upon responses from the caregiver. The NPI is administered at Baseline and at designated time points, e.g., the end of Weeks 12 and 24 (or upon early termination).

### DEFINITIONS

The term “treat” is used herein to mean to relieve or alleviate at least one symptom of a disease in a subject. For example, in relation to behavioral disorders, the term “treat” may mean to relieve or alleviate delusions, hallucinations, agitation/aggression, depression/dysphoria, anxiety, elation/euphoria, apathy/indifference, disinhibition, irritability/lability, aberrant motor activity, nighttime behavior, and appetite/eating changes. Within the meaning of the present invention, the term “treat” also denote to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject. Within the meaning of the present invention, the behavioral disorder is associated with a CNS disorder, including without limitation neurodegenerative diseases such as Alzheimer's disease (AD), Down's Syndrome and cerebrovascular dementia (VaD). Preferably, the behavioral disorder is associated with Alzheimer's disease (AD).

For example, as disclosed herein, a prophylactic administration of an 1-aminocyclohexane derivative can prevent or delay the onset of a behavioral disorder in a recipient subject at risk of developing such behavioral disorders associated with Alzheimer's disease as described in Example 1, infra. Similarly, according to the present invention, a therapeutic administration of an 1-aminocyclohexane derivative in combination with an AChEI, can prevent or delay the onset of development of clinical symptoms of behavioral disorders associated with Alzheimer's disease or even regression of symptoms as described in Example 2, infra.

Within the meaning of the present invention, the term “NMDA antagonist drugs” is used to refer to drugs that can suppress the triggering of NMDA receptor-mediated neuronal firings. Preferred NMDA antagonist drugs of the invention are 1-aminocyclohexane derivatives such as memantine and neramexane. These two exemplary compounds also have 5HT₃ antagonist activity and/or neuronal nicotinic receptor antagonist activity.

The term “analog” or “derivative” is used herein in the conventional pharmaceutical sense, to refer to a molecule that structurally resembles a reference molecule (such as 1-aminocyclohexane), but has been modified in a targeted and controlled manner to replace one or more specific substituents of the referent molecule with an alternate substituent, thereby generating a molecule which is structurally similar to the reference molecule. Synthesis and screening of analogs (e.g., using structural and/or biochemical analysis), to identify slightly modified versions of a known compound which may have improved or biased traits (such as higher potency and/or selectivity at a specific targeted receptor type, greater ability to penetrate mammalian blood-brain barriers, fewer side effects, etc.) is a drug design approach that is well known in pharmaceutical chemistry.

The term “1-aminocyclohexane derivative” is used herein to describe a compound which is derived from 1-aminocyclohexane (or an available derivative thereof, such as neramexane or memantine) in the process used to create a similar but slightly different drug.

The 1-aminocyclohexane derivatives of the present invention can be represented by the general formula (I): 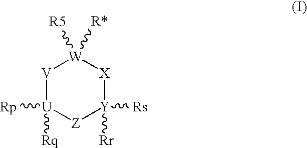
wherein:
R* is -(A)ₙ-(CR¹R²)ₘ—NR³R⁴,
   n+m=0, 1, or 2,
   A is selected from the group consisting of linear or branched lower alkyl (C₁-C₆),linear or branched lower alkenyl (C₂-C₆), and linear or branched lower alkynyl (C₂-C₆),
   R¹ and R² are independently selected from the group consisting of hydrogen, linear or branched lower alkyl (C₁-C₆), linear or branched lower alkenyl (C₂-C₆), linear or branched lower alkynyl (C₂-C₆) aryl, substituted aryl and arylalkyl,
   R³ and R⁴ are independently selected from the group consisting of hydrogen, linear or branched lower alkyl (C₁-C₆), linear or branched lower alkenyl (C₂-C₆), and linear or branched lower alkynyl (C₂-C₆), or together form alkylene (C₂-C₁₀) or alkenylene (C₂-C₁₀) or together with the N form a 3-7-membered azacycloalkane or azacycloalkene, including substituted (alkyl (C₁-C₆), alkenyl (C₂-C₆)) 3-7-membered azacycloalkane or azacycloalkene; or independently R³ or R⁴ may join with R^{p}, R^{q}, R^{r}, or R^{s} to form an alkylene chain —CH(R⁶)—(CH₂)ₜ—,
   wherein t=0 or 1 and the left side of the alkylene chain is attached to U or Y and the right side of the alkylene chain is attached to N and R⁶ is selected from the group consisting of hydrogen, linear or branched lower alkyl (C₁-C₆), linear or branched lower alkenyl (C₂-C₆), linear or branched lower alkynyl (C₂-C₆), aryl, substituted aryl and arylalkyl; or independently R³ or R⁴ may join with R⁵ to form an alkylene chain represented by the formula —CH₂—CH₂—CH₂—(CH₂)ₜ—, or an alkenylene chain represented by the formulae —CH═CH—CH₂—(CH₂)ₜ—, —CH═C═CH—(CH₂)ₜ— or —CH₂—CH═CH—(CH₂)ₜ—, wherein t=0 or 1, and the left side of the alkylene or alkenylene chain is attached to W and the right side of the alkylene ring is attached to N;
R⁵ is independently selected from the group consisting of hydrogen, linear or branched lower alkyl (C₁-C₆), linear or branched lower alkenyl (C₂-C₆), and linear or branched lower alkynyl (C₂-C₆), or R⁵ combines with the carbon to which it is attached and the next adjacent ring carbon to form a double bond,
R^{p}, R^{q}, R^{r}, and R^{s}, are independently selected from the group consisting of hydrogen, linear or branched lower alkyl (C₁-C₆), linear or branched lower alkenyl (C₂-C₆), linear or branched lower alkynyl (C₂-C₆), cycloalkyl (C₃-C₆) and aryl, substituted aryl and arylaklyl or R^{p}, R^{q}, R^{r}, and R^{s} independently may form a double bond with U or with Y or to which it is attached, or R^{p}, R^{q}, R^{r}, and R^{s} may combine together to represent a lower alkylene —(CH₂)ₓ— or a lower alkenylene bridge wherein x is 2-5, inclusive, which alkylene bridge may, in turn, combine with R⁵ to form an additional lower alkylene —(CH₂)_{y}— or a lower alkenylene bridge, wherein y is 1-3, inclusive,
the symbols U, V, W, X, Y, Z represent carbon atoms,
   and include optical isomers, diastereomers, polymorphs, enantiomers, hydrates, pharmaceutically acceptable salts, and mixtures of compounds within formula (I).

The ring defined by U-V-W-X-Y-Z is preferably selected from the group consisting of cyclohexane, cyclohex-2-ene, cyclohex-3-ene, cyclohex-1,4-diene, cyclohex-1,5-diene, cyclohex-2,4-diene, and cyclohex-2,5-diene.

Various salts and isomers (including stereoisomers and enantiomers) of memantine can be used. The term “salts” can include acid addition salts or addition salts of free bases. Examples of acids which may be employed to form pharmaceutically acceptable acid addition salts include inorganic acids such as hydrochloric, sulfuric, or phosphoric acid, and organic acids such as acetic, maleic, succinic, or citric acid, etc. All of these salts (or other similar salts) may be prepared by conventional means. The nature of the salt or isomer is not critical, provided that it is non-toxic and does not substantially interfere with the desired pharmacological activity. A preferred salt for the method of the present invention is memantine hydrochloride.

Non-limiting examples of 1-aminocyclohexane derivatives used according to the invention include the 1-aminoalkylcyclohexane derivatives selected from the group consisting of:
1-amino-1,3,5-trimethylcyclohexane,
1-amino-1(trans),3(trans),5-trimethylcyclohexane,
1-amino-1(cis),3(cis),5-trimethylcyclohexane,
1-amino-1,3,3,5-tetramethylcyclohexane,
1-amino-1,3,3,5,5-pentamethylcyclohexane (neramexane),
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-1,3,3,5,5-pentamethylcyclohexane,
N-ethyl-1-amino-1,3,3,5,5-pentamethyl-cyclohexane,
N-(1,3,3,5,5-pentamethylcyclohexyl) pyrrolidine,
3,3,5,5-tetramethylcyclohexylmethylamine,
1-amino-l-propyl-3,3,5,5-tetramethylcyclohexane,
1amino-1,3,3,5(trans)-tetramethylcyclohexane (axial amino group),
3-propyl-1,3,5,5-tetramethylcyclohexylamine semihydrate,
1-amino-1,3,5,5-tetramethyl-3-ethylcyclohexane,
1-amino-1,3,5-trimethylcyclohexane,
1-amino-1,3-dimethyl-3-propylcyclohexane,
1-amino-1,3(trans),5(trans)-trimethyl-3(cis)-propylcyclohexane,
1-amino-1,3-dimethyl-3-ethylcyclohexane,
1-amino-1,3,3-trimethylcyclohexane,
cis-3-ethyl-1(trans)-3(trans)-5-trimethylcyclohexamine,
1-amino-1,3(trans)-dimethylcyclohexane,
1,3,3-trimethyl-5,5-dipropylcyclohexylamine,
1-amino-1-methyl-3(trans)-propylcyclohexane,
1-methyl-3(cis)-propylcyclohexylamine,
1-amino-1-methyl-3(trans)-ethylcyclohexane,
1-amino-1,3,3-trimethyl-5(cis)-ethylcyclohexane,
1-amino-1,3,3-trimethyl-5(trans)-ethylcyclohexane,
cis-3-propyl-1,5,5-trimethylcyclohexylamine,
trans-3-propyl-1,5,5-trimethylcyclohexylamine,
N-ethyl-1,3,3,5,5-pentamethylcyclohexylamine,
N-methyl-l-amino-1,3,3,5.5-pentamethylcyclohexane,
1-amino-l-methylcyclohexane,
N,N-dimethyl-1-amino-1,3,3,5,5-pentamethylcyclohexane,
2-(3,3,5,5-tetramethylcyclohexyl)ethylamine,
2-methyl-l-(3,3,5,5-tetramethylcyclohexyl)propyl-2-amine,
2-(1,3,3,5,5-pentamethylcyclohexyl-l)-ethylamine semihydrate,
N-(1,3,3,5,5-pentamethylcyclohexyl)-pyrrolidine,
1-amino-1,3(trans),5(trans)-trimethylcyclohexane,
1-amino-1,3(cis),5(cis)-trimethylcyclohexane,
1-amino-(1R,SS)trans-5-ethyl-1,3,3-trimethylcyclohexane,
1-amino-(1S,SS)cis-5-ethyl-1,3,3-trimethylcyclohexane,
1-amino-1,5,5-trimethyl-3(cis)-isopropyl-cyclohexane,
1-amino-1,5,5-trimethyl-3(trans)-isopropyl-cyclohexane,
1-amino-1-methyl-3(cis)-ethyl-cyclohexane,
1-amino-1-methyl-3(cis)-methyl-cyclohexane,
1-amino-5,5-diethyl-1,3,3-trimethyl-cyclohexane,
1-amino-1,3,3,5,5-pentamethylcyclohexane,
1-amino-1,5,5-trimethyl-3,3-diethylcyclohexane,
1-amino-l-ethyl-3,3,5,5-tetramethylcyclohexane,
N-ethyl-l-amino-1,3,3,5,5-pentamethylcyclohexane,
N-(1,3,5-trimethylcyclohexyl)pyrrolidine or piperidine,
N-[1,3(trans),5(trans)-trimethylcyclohexyl]pyrrolidine or piperidine,
N-[1,3(cis),5(cis)-trimethylcyclohexyl]pyrrolidine or piperidine,
N-(1,3,3,5-tetramethylcyclohexyl)pyrrolidine or piperidine,
N-(1,3,3,5,5-pentamethylcyclohexyl)pyrrolidine or piperidine,
N-(1,3,5,5-tetramethyl-3-ethylcyclohexyl)pyrrolidine or piperidine,
N-(1,5,5-trimethyl-3,3-diethylcyclohexyl)pyrrolidine or piperidine,
N-(1,3,3-trimethyl-cis-5-ethylcyclohexyl)pyrrolidine or piperidine,
N-[(1S,SS)cis-5-ethyl-1,3,3-trimethylcyclohexyl]pyrrolidine or piperidine,
N-(1,3,3-trimethyl-trans-5-ethylcyclohexyl)pyrrolidine or piperidine,
N-[(1R,SS)trans-5-ethyl,3,3-trimethylcyclohexyl]pyrrolidine or piperidine,
N-(1-ethyl-3,3,5,5-tetramethylyclohexyl)pyrrolidine or piperidine,
N-(1-propyl-3,3,5,5-tetramethylcyclohexyl)pyrrolidine or piperidine,
N-(1,3,3,5,5-pentamethylcyclohexyl)pyrrolidine,
   their optical isomers, diastereomers, enantiomers, hydrates, their pharmaceutically acceptable salts, and mixtures thereof.

Neramexane (1-amino-1,3,3,5,5-pentamethylcyclohexane) is disclosed, e.g., in U.S. patent application Ser. No. 09/597,102 and U.S. Pat. No. 6,034,134.

Certain 1-aminocyclohexane derivatives of general formula (I) including the case where three axial alkyl substituent, e.g., R^{p}, R^{r} and R⁵ all together form a bridgehead to yield compounds (so called 1-aminoadamantanes) illustrated by the formulae IIb-IId below:

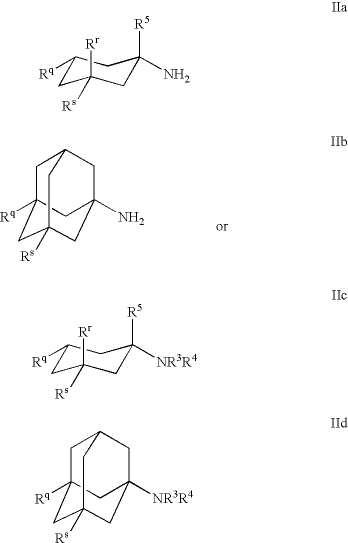

Certain 1-aminocyclohexane derivatives of forumula (I) wherein n+m=0, U, V, W, X, Y and Z form a cyclohexane ring, and one or both of R³ and R⁴ are independently joined to said cyclohexane ring via alkylene bridges formed through R^{p}, R^{q}, R^{r}, R^{s} or R⁵ are represented by the following formulae IIIa-IIIc: 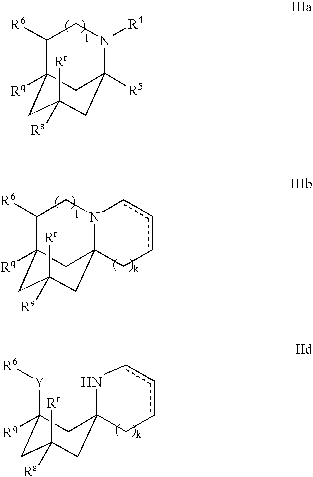
where R^{q}, R^{r}, R^{s}, R^{r} and R⁵ are as defined above for formula (I), R⁶ is hydrogen, linear or branched lower alkyl (C₁-C₆), linear or branched lower alkenyl (C₂-C₆), linear or branched lower alkynyl (C₂-C₆), aryl, substituted aryl or arylalkyl Y is saturated or may combine with R⁶ to form a carbon-hydrogen bond with the ring carbon to which it is attached, I=0 or 1 and k=0, 1 or 2 and [symbol] represents a single or double bond.

Non-limiting examples of 1-aminocyclohexane derivatives used according to the invention include 1-amino adamantane and its derivatives selected from the group consisting of:
1-amino-3-phenyl adamantane,
1-amino-methyl adamantane,
1-amino-3,5-dimethyl adamantane (memantine),
1-amino-3-ethyl adamantane,
1-amino-3-isopropyl adamantane,
1-amino-3-n-butyl adamantane,
1-amino-3,5-diethyl adamantane,
1-amino-3,5-diisopropyl adamantane,
1-amino-3,5-di-n-butyl adamantane,
1-amino-3-methyl-5-ethyl adamantane,
1-N-methylamino-3,5-dimethyl adamantane,
1-N-ethylamino-3,5-dimethyl adamantane,
1-N-isopropyl-amino-3,5-dimethyl adamantane,
1-N,N-dimethyl-amino-3,5-dimethyl adamantane,
1-N-methyl-N-isopropyl-amino-3-methyl-5-ethyl adamantane,
1-amino-3-butyl-5-phenyl adamantane,
1-amino-3-pentyl adamantane,
1-amino-3,5-dipentyl adamantane,
1-amino-3-pentyl-5-hexyl adamantane,
1-amino-3-pentyl-5-cyclohexyl adamantane,
1-amino-3-pentyl-5-phenyl adamantane,
1-amino-3-hexyl adamantane,
1-amino-3,5-dihexyl adamantane,
1-amino-3-hexyl-5-cyclohexyl adamantane,
1-amino-3-hexyl-5-phenyl adamantane,
1-amino-3-cyclohexyl adamantane,
1-amino-3,5-dicyclohexyl adamantane,
1-amino-3-cyclohexyl-5-phenyl adamantane,
1-amino-3,5-diphenyl adamantane,
1-amino-3,5,7-trimethyl adamantane,
1-amino-3,5-dimethyl-7-ethyl adamantane,
1-amino-3,5-diethyl-7-methyl adamantane,
1-N-pyrrolidino and 1-N-piperidine derivatives,
1-amino-3-methyl-5-propyl adamantane,
1-amino-3-methyl-5-butyl adamantane,
1-amino-3-methyl-5-pentyl adamantane,
1-amino-3-methyl-5-hexyl adamantane,
1-amino-3-methyl-5-cyclohexyl adamantane,
1-amino-3-methyl-5-phenyl adamantane,
1-amino-3-ethyl-5-propyl adamantane,
1-amino-3-ethyl-5-butyl adamantane,
1-amino-3-ethyl-5-pentyl adamantane,
1-amino-3-ethyl-5-hexyl adamantane,
1-amino-3-ethyl-5-cyclohexyl adamantane,
1-amino-3-ethyl-5-phenyl adamantane,
1-amino-3-propyl-5-butyl adamantane,
1-amino-3-propyl-5-pentyl adamantane,
1-amino-3-propyl-5-hexyl adamantane,
1-amino-3-propyl-5-cyclohexyl adamantane,
1-amino-3-propyl-5-phenyl adamantane,
1-amino-3-butyl-5-pentyl adamantane,
1-amino-3-butyl-5-hexyl adamantane,
1-amino-3-butyl-5-cyclohexyl adamantane,
   their optical isomers, diastereomers, enantiomers, hydrates, N-methyl, N,N-dimethyl, N-ethyl, N-propyl derivatives, their pharmaceutically acceptable salts, and mixtures thereof.

Memantine (1-amino-3,5-dimethyl adamantane), and pharmaceutically acceptable salts thereof, for example, is the subject matter of U.S. Pat. Nos. 4,122,193 and 4,273,774.

The 1-amino adamantane derivatives of formulae IIb and IId, including memantine, are generally prepared by alkylation of halogenated adamantanes, preferably bromo- or chloroadamantanes. The di- or tri-substituted adamantanes are obtained by additional halogenation and alkylation procedures. The amino group is introduced either by oxidation with chromiumtrioxide and bromination with HBr or bromination with bromine and reaction with formamide followed by hydrolysis. The amino function can be alkylated according to generally-accepted methods. Methylation can, for example, be effected by reaction with chloromethyl formate and subsequent reduction. The ethyl group can be introduced by reduction of the respective acetamide. For more details on synthesis see, e.g., U.S. Pat. Nos. 5,061,703 and 6,034,134. Additional synthetic techniques for the foregoing compounds can be found in U.S. provisional applications Ser. No. 60/350,974 filed Nov. 7, 2001, Ser. No. 60/337,858 filed Nov. 8, 2001, and Ser. No. 60/366,386 filed Mar. 21, 2002, all incorporated by reference, as well as in the Synthesis Examples below.

According to the invention, the 1-aminocyclohexane derivatives of formula (I) may be applied as such or used in the form of their pharmaceutically-acceptable salts including, for example, the acid addition salts such as hydrochlorides, hydrobromides, sulfates, acetates, succinates or tartrates, or their acid addition salts with fumaric, maleic, citric, or phosphoric acids.

In addition, using methods known to those skilled in the art, analogs and derivatives of the compounds of the invention can be created which have improved therapeutic efficacy, i.e., higher potency and/or selectivity at a specific targeted receptor type, either greater or lower ability to penetrate mammalian blood-brain barriers (e.g., either higher or lower blood-brain barrier permeation rate), fewer side effects, etc.

Various salts and isomers (including stereoisomers and enantiomers) of the drugs listed herein can be used. The term “salts” can include addition salts of free acids or free bases. Examples of acids which may be employed to form pharmaceutically acceptable acid addition salts include inorganic acids such as hydrochloric, sulfuric, or phosphoric acid, and organic acids such as acetic, maleic, succinic, or citric acid, etc. All of these salts (or other similar salts) may be prepared by conventional means. The nature of the salt or isomer is not critical, provided that it is non-toxic and does not substantially interfere with the desired pharmacological activity.

The term “acetylcholinesterase inhibitor” or “AChEI” is used herein to refer to a drug that enhances function of cholinergic neurons by inhibiting the catabolic enzyme acetylcholinesterase (AChE). The term encompasses reversible, pseudo-reversible and irreversible AChEIs as well as AChEIs that selectively inhibit AChE, and AChEIs, that are less selective (e.g., also target butyrylcholinesterase, BuChE). Preferably, AChEIs useful in the methods and compositions of the present invention are reversible or pseudo-reversible. Specific examples of AChEIs useful in the methods and compositions of the present invention include, but are not limited to, tacrine (THA; 1,2,3,4-tetrahydro-9-aminoacridine hydrochloride), donepezil, galantamine, rivastigmine, huperzine A, zanapezil, ganstigmine, phenserine, phenethylnorcymserine (PENC), cymserine, thiacymserine, SPH 1371 (galantamine plus), ER 127528, RS 1259, and F3796.

The term “combination” applied to active ingredients is used herein to define a single pharmaceutical composition (formulation) comprising both drugs of the invention (i.e., an 1-aminocyclohexane derivative and an AChEI) or two separate pharmaceutical compositions (formulations), each comprising a single drug of the invention (i.e., an 1-aminocyclohexane derivative or an AChEI), to be administered conjointly.

Within the meaning of the present invention, the term “conjoint administration” is used to refer to administration of the 1-aminocyclohexane derivative and AChEI simultaneously in one composition, or simultaneously in different compositions, or sequentially. For the sequential administration to be considered “conjoint”, however, the 1-aminocyclohexane derivative and AChEI must be administered separated by a time interval that still permits the resultant beneficial effect for treating, preventing, arresting, delaying the onset of and/or reducing the risk of developing a behavioral disorder associated with a central nervous system (CNS) disorder in a mammal. For example, the 1-aminocyclohexane derivative and AChEI must be administered on the same day (e.g., each—once or twice daily), preferably within an hour of each other, and most preferably simultaneously.

The term “therapeutically effective” applied to dose or amount refers to that quantity of a compound or pharmaceutical composition that is sufficient to result in a desired activity upon administration to a mammal in need thereof. As used herein with respect to the pharmaceutical compositions comprising an 1-aminocyclohexane derivative, the term “therapeutically effective amount/dose” is used interchangeably with the term “neurologically effective amount/dose” and refers to the amount/dose of a compound or pharmaceutical composition that is sufficient to produce an effective neurological response, i.e., improvement of a behavioral disorder associated with a CNS disorder, upon administration to a mammal.

The term “subthreshold”, referring to the amount of an active ingredient, means an amount inadequate to produce a response, i.e., an amount below the minimum effective amount. The term “suboptimal” in the same context means an amount of an active ingredient that produces a response but not to its full extent, which would be achieved with a higher amount.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “carrier” applied to pharmaceutical compositions of the invention refers to a diluent, excipient, or vehicle with which an active compound (e.g., an 1-aminocyclohexane derivative) is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by E. W. Martin, 18^{th} Edition.

The term “subject” as used herein refers to a mammal (e.g., rodent such as mouse or rat). In particular, the term refers to humans presenting with a behavioral disorder associated with a CNS disorder or brain injury, or one of the foregoing underlying conditions discussed in connection with agitation, in the Background Section, above.

A “responder” is defined as a patient for whom the change from baseline (no treatment) on a clinical subscale, e.g., NPI scale, improves. For instance, a patient having a baseline NPI of greater than 4, which decreases upon memantine administration to a significantly lower number, or to a 4 or below, is a responder. A responder in terms of SIB scale means a subject for whom the score increases with memantine relative to a patient not being administered memantine. A responder in terms of the ADCS-ADL scale refers to a patient for whom the score increases with memantine relative to a patient not being administered memantine, or who demonstrates an improvement of any symptom or behavior. The modified ADCS-ADL₁₉ scale, has a scoring range of 0 to 54, with the lower scores indicating greater functional impairment. A responder in terms of CIBIC-plus scale is defined as a patient for whom CIBIC-plus equals to “Markedly improved”, or “Moderately improved”, or “Minimally improved”, or “no change” after memantine administration. A score of 1-3 indicates improvement, a score of 4 indicates no change, and scores of 5-7 indicate worsening of impairment.

In a preferred embodiment, a responder according to the present invention is a patient for whom the behavioral symptoms improve with treatment of a 1-aminocyclohexane compared with an untreated control population. For example, a responder would be a patient exhibiting a reduction in the number of incidents, a reduction in the degree of severity, or an absence, of delusions, hallucinations, agitation/aggression, depression/dysphoria, anxiety, elation/euphoria, apathy/indifference, disinhibition, irritability/lability, aberrant motor activity, nighttime behavior, and appetite/eating changes.

The term “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

### Pharmaceutical Formulations and Administration

In conjunction with the methods of the present invention, also provided are pharmaceutical compositions comprising a therapeutically effective amount of an 1-aminocyclohexane derivative (such as memantine or neramexane) alone or in combination with a therapeutically effective amount of an acetylcholinesterase inhibitor (AChEI) (such as galantamine, tacrine, donepezil, or rivastigmine) and/or further in combination with an additional active ingredient, e.g., an antipsychotic in the event the behaviorial disorder is agitation. The compositions of the invention further can comprise a carrier or excipient (all pharmaceutically acceptable). Said combination of 1-aminocyclohexane derivative and an AChEI or antipsychotic can be either formulated as a single composition or as two separate compositions, which can be administered conjointly. Preferably, they are administered simultaneously. The compositions can be formulated for once-a-day administration or twice-a-day administration. Thus, the aminocyclohexane derivative can be administered b-i-d and the AChEI can be administered b-i-d as one or as two different compositions for each administration. In another embodiment, the aminocyclohexane derivative can be administered b-i-d and the AChEI can be administered once a day (or vice-versa). In a further embodiment, the aminocyclohexane derivative and AChEI can each be administered once a day as one or as two different compositions.

Similar administration regimens can be used when an antipsychotic in combination with an aminocyclohexane derivative, or both an amino-cyclohexane derivative and an AChEI. Antipsychotics are typically administered in various doses depending on the drug. The following are typical doses of atypical antipsychotics: clozapine-300-600 mg/day; olanzapine-15-20 mg/day; quetiapine-400-600 mg/day; risperidone 4-8 mg/day; ziprasidone-80-160 mg/day.

In the disclosed compositions, preferably, the 1-aminocyclohexane derivative or the 1-aminocyclohexane derivative/AChEI combination are present in therapeutically effective amounts. The optimal therapeutically effective amount should be determined experimentally, taking into consideration the exact mode of administration, from in which the drug is administered, the indication toward which the administration is directed, the subject involved (e.g., body weight, health, age, sex, etc.), and the preference and experience of the physician or veterinarian in charge. As disclosed herein, for human administration, both the 1-aminocyclohexane derivatives and AChEIs are administered in suitable form in doses ranging from about 1 to 200 mg per day for each drug. More specifically, the 1-aminocyclohexane derivatives are preferably administered at doses 5-60 mg/day, and especially 10-40 mg/day; the AChEIs are preferably administered at doses 1-40 mg/day, and especially 5-24 mg/day. It may also be desirable in certain cases to administer one or the other of the active ingredients in a suboptional or subthreshold amount, and such administration would also be within the invention.

The invention also provides a method for preparing pharmaceutical compositions comprising admixing an 1-aminocyclohexane derivative alone or in combination with an AChEI in therapeutically effective amounts, and optionally one or more physiologically acceptable carriers and/or excipients and/or auxiliary substances.

Administration

The active agents of the present invention may be administered orally, topically, parenterally, or mucosally (e.g., buccally, by inhalation, or rectally) in dosage unit formulations containing conventional non-toxic pharmaceutically acceptable carriers. It is usually desirable to use the oral route. The active agents may be administered orally in the form of a capsule, a tablet, or the like, or as a semi-solid or liquid formulation (see Remington's Pharmaceutical Sciences, Mack 5 Publishing Co., Easton, Pa.). The orally administered medicaments may be administered in the form of a time-controlled release vehicle, including diffusion-controlled systems, osmotic devices, dissolution-controlled matrices, and erodible/degradable matrices. Usually the 1-aminocyclohexane derivative, i.e., memantine, will constitute between 0.1 and 99% by weight of the formulation, more specifically between 0.5 and 20% by weight for formulations intended for injection and between 0.2 and 50% by weight for formulations suitable for oral administration.

For oral administration in the form of a tablet or capsule, the active drug component can be combined with a non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like. For oral administration in liquid form, the drug components can be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) can also be added to stabilize the dosage forms.

The tablets can be coated by methods well known in the art. The cores, prepared as described above, may also be coated with a concentrated sugar solution which may contain e.g., gum arabic, gelatine, talcum, titanium dioxide, and the like. Alternatively, the tablets can be coated with a polymer known to a person skilled in the art, wherein the polymer is dissolved in a readily volatile organic solvent or mixture of organic solvents. Dyestuffs may be added to these coatings in order to readily distinguish between tablets containing different active substances or different amounts of the active compounds.

For the formulation of soft gelatin capsules, the active substances may be admixed with e.g., a vegetable oil or poly-ethylene glycol. Hard gelatin capsules may contain granules of the active substances using either the above mentioned excipients for tablets e.g., lactose, saccharose, sorbitol, mannitol, starches (e.g., potato starch, corn starch or amylopectin), cellulose derivatives or gelatine. Also liquids or semisolids of the drug can be filled into hard gelatine capsules.

The compositions of the invention can be also introduced in microspheres or microcapsules, e.g., fabricated from polyglycolic acid/lactic acid (PGLA) (see, e.g., U.S. Pat. Nos. 5,814,344; 5,100,669 and 4,849,222; PCT Publications No. WO95/11010 and WO93/07861). Liquid preparations for oral administration can take the form of, for example, solutions, syrups, emulsions or suspensions, or they can be presented as a dry product for reconstitution with water or other suitable vehicle before use. Preparations for oral administration can be suitably formulated to give controlled or postponed release of the active compound. A particular example of an oral time-controlled release pharmaceutical formulation is described in U.S. Pat. No. 5,366,738.

Liquid formulations for oral application may be in the form of syrups or suspensions, for example, solutions containing from about 0.2% to about 20% by weight of the active substances herein described, the balance being sugar and mixture of ethanol, water, glycerol and propylene glycol. Optionally such liquid formulations may contain coloring agents, flavoring agents, saccharine and carboxymethyl-cellulose as a thickening agent or other excipients known to a person skilled in the art.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines, as is well known.

Drugs of the invention may also be delivered by the use of monoclonal antibodies as individual carriers to which the compound molecules are coupled. Active drugs may also be coupled with soluble polymers as targetable drug carriers. Such polymers can include polyvinyl-pyrrolidone, pyran copolymer, polyhydroxy-propyl methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

For administration by inhalation, the therapeutics according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The formulations of the invention can be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection can be presented in unit dosage form, e.g., in ampoules or in multi-dose containers, with an added preservative. The compositions can take such forms as excipients, suspensions, solutions, or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water, before use.

Solutions for parenteral applications by injection can be prepared in an aqueous solution of a water-soluble pharmaceutically acceptable salt of the active substances, preferably in a concentration of from about 0.5% to about 10% by weight. These solutions may also contain stabilizing agents and/or buffering agents and may conveniently be provided in various dosage unit ampoules.

Dosage units for rectal application can be solutions or suspensions or can be prepared in the form of suppositories or retention enemas comprising the active substances in a mixture with a neutral fatty base, or gelatin rectal capsules comprising the active substances in admixture with vegetable oil or paraffin oil.

As disclosed herein, an 1-aminocyclohexane derivative, optionally with an AChEI can be mixed with excipients which are pharmaceutically acceptable and compatible with the active ingredients. In addition, if desired, the preparations may also include minor amounts of auxiliary substances such as wetting or emulsifying agents, pH buffering agents, and/or agents that enhance the effectiveness of the pharmaceutical composition. These auxiliary molecules can be delivered systemically or locally as proteins or by expression of a vector that codes for expression of the molecule. The techniques described above for the delivery of 1-aminocyclohexane derivatives and AChEIs can also be employed for the delivery of auxiliary molecules.

Although the active agents of the present invention may be administered in divided doses, for example, two or three times daily, a single daily dose of each of the 1-aminocyclohexane derivative and AChEI is preferred, with a single daily dose of both agents in one composition or in two separate compositions administered simultaneously being most preferred.

The instant invention also encompasses a process for preparing pharmaceutical compositions comprising combining an 1-aminocyclohexane derivative and/or an AChEI with a pharmaceutically acceptable carrier and/or excipient.

Suitable daily doses of the memantine for the therapeutic treatment of humans are about 0.01-10 mg/kg bodyweight on peroral administration and 0.001-10 mg/kg bodyweight on parenteral administration.

Preferred specific amounts of the 1-aminocyclohexane derivative which may be used in unit dosage amounts of the invention include, for example, 5 mg, 10 mg, 15 mg, and 20 mg for memantine and 5 mg, 10 mg, 20 mg, 30 mg, and 40 mg for neramexane. Preferred specific amounts of the AChEI which may be used in unit dosage amounts of the invention include, for example, 1.5 mg, 3 mg, 4.5 mg, and 6 mg for rivastigmine, 4 mg, 8 mg and 12 mg for galantamine, and 5 mg and 10 mg for donepezil.

In one embodiment, 5 or 10 mg film-coated memantine tablets can be administered twice a day for a dosage range of 10-40 mg/day. However, lower and higher dosages can be and have been administered within the range of 5-100 mg/day and the broader range of 5-200 mg/day.

The invention also provides a pharmaceutical pack or kit comprising one or more containers containing one or more of the ingredients of the formulations of the invention. In a related embodiment, the present invention provides a kit for the preparation of the pharmaceutical compositions of the invention, said kit comprising an 1-aminocyclohexane derivative in a first container, and an AChEI in a second container, and, optionally, instructions for admixing the two drugs and/or for administration of the compositions. Each container of the kit may also optionally include one or more physiologically acceptable carriers and/or excipients and/or auxiliary substances. Associated with such container(s) can be a notice in the form prescribed by a governmental agency regulating the manufacture, use or sale of pharmaceuticals or biological products, which notice reflects approval by the agency of manufacture, use or sale for human administration.

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions of the invention formulated in a compatible pharmaceutical carrier may also be prepared, placed in an appropriate container, and labeled for treatment of an indicated condition.

### Effective Dose Determination and Safety Evaluations

According to the methods of the present invention, the pharmaceutical compositions described herein are administered to a patient at therapeutically effective doses, preferably, with minimal toxicity. The Section entitled “Definitions” provides definitions for the terms “neurologically effective dose” and “therapeutically effective dose”. Preferably, the 1-aminocyclohexane derivative alone or in combination with the AChEI are each used at a dosage which, when combined, provide an enhanced effect, most preferably, an effect not observed upon administration of each agent alone.

The efficacy of the 1-aminocyclohexane derivatives of the invention can be determined using such in vitro pharmacological tests as measurements of displacement of [³H]MK-801 binding in rat or human brain tissue, blocking of NMDA receptor channels in cultured neurones and heterologous expression systems, anticonvulsive effects in vivo, correlation between channel-blocking and anticonvulsive action, protection against cerebral ischemia, protection against NMDA-induced mortality, etc. (see, e.g., U.S. Pat. No. 5,061,703).

The efficacy of the AChEIs of the invention can be determined in vitro using such well-known methods as the spectrophotometric assay of AChE activity described by Ellman et al. (Biochem. Pharmacol., 7: 86-95, 1961; see also Wenk et al., Life Sci., 2000, 66:1079-1083).

Following methodologies which are well-established in the art, effective doses and toxicity of the compounds and compositions of the instant invention, which performed well in in vitro tests, are then determined in preclinical studies using small animal models (e.g., mice or rats) in which both the 1-aminocyclohexane derivatives and AChEIs has been found to be therapeutically effective and in which these drugs can be administered by the same route proposed for the human clinical trials.

For any pharmaceutical composition used in the methods of the invention, the therapeutically effective dose can be estimated initially from animal models to achieve a circulating plasma concentration range that includes the IC₅₀ (i.e., the concentration of the test compound which achieves a half-maximal inhibition of NMDA receptor activity and/or AChE enzymatic activity in the relevant areas of the brain). Dose-response curves derived from animal systems are then used to determine testing doses for the initial clinical studies in humans. In safety determinations for each composition, the dose and frequency of administration should meet or exceed those anticipated for use in the clinical trial.

As disclosed herein, the dose of the components in the compositions of the present invention is determined to ensure that the dose administered continuously or intermittently will not exceed an amount determined after consideration of the results in test animals and the individual conditions of a patient. A specific dose naturally varies depending on the dosage procedure, the conditions of a patient or a subject animal such as age, body weight, sex, sensitivity, feed, dosage period, drugs used in combination, and seriousness of the disease. The appropriate dose and dosage times under certain conditions can be determined by the test based on the above-described indices but may be refined and ultimately decided according to the judgment of the practitioner and each patient's circumstances (age, general condition, severity of symptoms, sex, etc.) according to standard clinical techniques. As disclosed herein, an appropriate dose of an 1-aminocyclohexane derivative is generally in the range of 0.05-1.00 mg per kg of body weight, and an appropriate dose of or an AChEI is generally in the range of 0.015-0.57 mg per kg of the body weight.

Toxicity and therapeutic efficacy of the compositions of the invention can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀. Compositions that exhibit large therapeutic indices are preferred.

The data obtained from animal studies can be used in formulating a range of doses for use in humans. The therapeutically effective doses of 1-aminocyclohexane derivatives and AChEIs in humans lay preferably within a range of circulating concentrations that include the ED₅₀ with little or no toxicity. For example, such therapeutically effective circulating concentration for memantine is 1 μM and for tacrine (AChEI) is 8-30 nM (Roberts et al., Eur. J. Clin. Pharmacol., 1998, 54: 721-724). The dosage can vary within this range depending upon the dosage form employed and the route of administration utilized. Ideally, a single dose of each drug should be used daily.

The drug combination of the invention is not only highly effective at relatively low doses but also possesses low toxicity and produces few side effects. Indeed, the only common side effect for the AChEIs of the invention is minor gastric irritation (e.g., in nausea, diarrhea, or vomiting), while the most common side effect resulting from the use of 1-aminocyclohexane derivatives of the invention is a minor motor and cognitive impairment (reflected, e.g., in nausea, vomiting, dizziness, or confusion).

### THERAPY EXAMPLES

The following Therapy Examples illustrate the invention without limiting its scope.

### Therapy Example 1

### Effect of Memantine on Behavioral Outcomes in Patients Suffering from Moderate to Severe Alzheimer's Disease

Patients suffering from moderate to severe AD were randomized to placebo or 20 mg memantine daily for 28 weeks. Primary efficacy variables were the Clinician's Interview-Based Impression of Change plus caregiver input (CIBIC-Plus) and the Alzheimer's Disease Cooperative Study Activities of Daily Living Inventory modified for severe dementia (ADCS-ADL). Secondary efficacy variables included the Severe Impairment Battery (SIB) and Neuropsychiatric Inventory (NPI). Treatment differences between baseline and endpoint were assessed. Incomplete observations were imputed using the most recent previous observation (last observation carried forward—LOCF). Results were also analyzed using only the observed values, where missing values were not replaced (Observed Cases (OC) analysis). AD patients (N=252), (67 percent female, mean age=76 years), were randomized from 32 U.S. centers. Of these, 181 completed the study (72 percent) and were evaluated at week 28. Seventy-one patients discontinued treatment prematurely (42 placebo, 29 memantine). Change on the CIBIC-Plus favored memantine over placebo (P=0.06 LOCF, P=0.03 OC) Memantine-treated patients deteriorated less than placebo treated patients on the ADCS-ADL (P=0.02 LOCF, P=0.003 OC) and the SBI (P=<0.001 LOCF, P=0.002 OC). NPI change scores at week 28 were statistically significant. Depending on the type of analysis, this benefit was found in two domains: agitation (p=0.008 LOCF) and delusion (p=0.04 LOCF) or for agitation, only (p=0.023 OC). The finding for agitation also occurred in the dichotomised analyses for memantine-treated patients with and without behavioural symptoms at baseline. Memantine was not associated with significant adverse events. This study supports the antiglutamatergic treatment approach in moderate to severe AD, a phase associated with patient distress and caregiver burden, for which other treatments are not available and the treatment of behavioral disorders associated therewith.

Methods

Patients

Community residing patients at least 50 years old with probable AD according to Diagnostic and Statistical Manual of Mental Disorders (DSM-IV) (American Psychiatric Association. Diagnostic and statistical manual of mental disorders. 4^{th} ed. Washington: American Psychiatric Association; 1994.) and National Institute of Neurologic and Communicative Disorders and Stroke and AD and Related Disorders Association (NINCDS-ADRDA) criteria (McKhann G, Drachman D, Folstein M, Katzman R, Price D, Stadlan E M, Clinical diagnosis of Alzheimer's disease: report of the NINCDS-ADRDA Work Group under the auspices of Department of Health and Human Services Task Force on Alzheimer's Disease. Neurology 1984;34:939-44) were recruited. Eligibility criteria included: baseline Mini-Mental State Examination (MMSE) scores of 3-14 (Folstein M F, Folstein, S E, McHugh, P R, Mini-mental state. A practical method for grading the cognitive state of patients for the clinician. J Psychiat Res 1975;12:189-98.), stage 5 or 6 on the Global Deterioration Scale (GDS) (Reisberg B, Ferris S H, de Leon M J, Crook T, The Global Deterioration Scale for assessment of primary degenerative dementia. Am J Psychiatry 1982;139:1136-9.), and a stage of 6a or greater on the Functional Assessment Staging (FAST) (Sclan S G, Reisberg B, Functional assessment staging (FAST) in Alzheimer's disease: reliability, validity, and ordinality. Int Psychogeriatr 1992;4 Suppl 1:55-69.) instrument, signifying the presence of dementia-related deficits in the ability to perform one or more basic activities of daily living. Patients had reliable caregivers and recent (within 12 months) computed tomography (CT) or magnetic resonance imaging (MRI).

Patients with vascular dementia, dementias or significant neurological disease secondary to conditions other than AD, major depressive disorder, or a score>4 on the modified Hachinski lschemic Rating Scale (HIS) (Rosen W G, Terry R D, Fuld P A, Katzman R, Peck A. Pathological verification of ischemic score in differentiation of dementias. Ann Neurol 1980;7:486-8.) were excluded. Patients with clinically significant concomitant medical co-morbidity or laboratory abnormalities were excluded as were patients receiving specific concomitant medications (anticonvulsants, anti-Parkinsonian agents, hypnotics, anxiolytics, neuroleptics, cholinomimetics, or any other investigational compounds). Patients stabilized on antidepressants for at least 2 months were eligible, and chloral hydrate could be used as a sedative/hypnotic, but not within 24 hours of an assessment.

Study Design

In this 28-week, double-blind, parallel-group study, patients were randomized to receive either memantine (20 mg/day) or identical placebo. Randomization was stratified by site using RANCODE version 3.1 and in blocks of 4, with sites blinded to the randomization process. Patients were discontinued from their randomized treatment if continuation represented a medical risk in the opinion of the study physician, or if they declined ongoing participation. Premature withdrawals were asked to complete endpoint measures at early termination and to return for a “retrieved dropout visit” at 28 weeks which included all endpoint assessments.

Efficacy Variables

Pre-specified efficacy variables were: (1) Clinician's Interview-Based Impression of Change plus caregiver input (CIBIC-Plus) global score, (2) change from baseline in the Alzheimer's Disease Cooperative Study Activities of Daily Living Inventory (ADCS-ADL) modified for more severe dementia (ADCS-ADLsev), (3) the SIB (Severe Impairment Battery) and (4) the Neuropsychiatric Inventory (NPI). See above for a description of these clinical assessment scales. Assessments were conducted at baseline, mid-study (Week 12), and at the end of treatment (Week 28) or at early termination, with a 28-week retrieved drop-out visit when possible.

Statistical Procedures and Populations for Analysis

The predefined efficacy analysis was based on those randomized patients who received at least one post-baseline assessment. This analysis included both study completers and those who discontinued their randomized treatment prematurely. For the latter, the week 28 efficacy observation was imputed by the last available observation (LOCF) (Gillings D, Koch G. The application of the principle of intent-to-treat to the analysis of clinical trials. Drug Inf J 1991;25:411-424.). Additional analyses were also performed to adjust for missing values. An observed-cases (OC) analysis was also undertaken based on all randomized patients available for evaluation at week 28. Efficacy outcomes were analyzed by the Wilcoxon-Mann-Whitney test for independent samples, using change from baseline. There were no interim analyses. The pre-specified responder group was defined as patients who improved or exhibited no deterioration on the CIBIC-Plus and who improved or did not deteriorate on either the ADCS-ADLsev or SIB. All patients were included in the safety analysis. All reported p-values are two-sided.

Results

Study Population

Of 345 patients, 252 were randomized. Seventy-one of the patients (42 placebo, 29 memantine) discontinued their randomized treatment prior to week 28 and the remaining 181 completed the double-blind portion of the study. Five subjects were excluded from the ADCS-ADL (CIBIC-plus) analyses because of no post-baseline assessments. Only 5 of the 71 discontinued subjects returned for a week 28 retrieved drop-out visit. Premature discontinuations were due to adverse events in 17 percent of placebo-treated and 10 percent of memantine-treated patients.

The mean baseline MMSE score for this study population was 7.9.

### Example 1

### Demographic Characteristics

| | Placebo | Memantine |
|---|---|---|
| Safety Population | (n = 126) | (n = 126) |
| Male/Female | 47/79 | 35/91 |
| Mean age, yrs | 76.3 | 75.9 |
| Race-Caucasian, n (%) | 115 (91%) | 112 (89%) |
| Mean MMSE score (SD) | 8.05 (3.6) | 7.72 (3.7) |
| Mean SIB score (SD) | 68.3 (20.8) | 65.9 (22.5) |
| Mean ADCS-ADL score (SD) | 27.4 (10.9) | 26.8 (9.2) |

MMSE = Mini-Mental State Examination
SIB = Severe Impairment Battery
ADCS-ADL = Alzheimer's Disease Cooperative Study - Activities of Daily Living Inventory

Efficacy

Baseline scores, and results based on the LOCF and OC analyses for the efficacy variables were evaluated. The CIBIC-Plus ratings at endpoint (mean difference, 0.3, P=0.06) and week 28 (mean difference, 0.4, P=0.03) supported the effectiveness of memantine.

ADCS-ADL total scores at baseline were similar for both groups (27.4 for placebo and 26.8 for memantine). At endpoint and week 28, there was significantly less deterioration in the memantine group (LOCF, mean difference, −2.1, P=0.02, and OC, mean difference, −3.4, P=0.003) compared to placebo.

SIB total scores at baseline were similar for both groups (27.4 for placebo and 26.8 for memantine). At endpoint and week 28, there was significantly less deterioration in the memantine group (LOCF, mean difference, −2.1, P=0.02, and OC, mean difference, −3.4, P=0.003) compared to placebo.

NPI data were analysed by total score and domains using all patients and then dichotomised for patients with and without behavioural symptoms at baseline. The NPI total score indicated a benefit for memantine-treated patients compared to placebo-treated patients. Depending on the type of analysis, this benefit was found in two domains: agitation (p=0.008 LOCF) and delusion (p=0.04 LOCF), or for agitation, only (p=0.023 OC, cf. Table 1). The finding for agitation also occurred in the dichotomised analyses for memantine-treated patients with and without behavioral symptoms at baseline. Consistent with the results of the agitation domain of the NPI was the observation that significantly fewer memantine-treated patients experienced an adverse event of agitation compared to placebo-treated patients (18% vs 32%, respectively; p=0.02), despite more memantine-treated patients than placebo-treated patients having behavioural symptoms at baseline.

TABLE 1
Change (baseline to week 28) in NPI by domain with
Alzheimer's patients treated with memantine.
NPI by domain, ITT, OC

| | | | p value for |
|---|---|---|---|
| DOMAIN | placebo | memantine | change |
| Delusions | 0.33 | −0.38 | 0.085410 |
| Hallucinations | −0.04 | 0.47 | 0.252547 |
| Agitation/Aggression | 0.87 | −0.01 | 0.023283 |
| Depression/Dysphoria | 0.63 | 0.00 | 0.398710 |
| Anxiety | 0.27 | 0.51 | 0.509252 |
| Elation/Euphoria | 0.25 | 0.18 | 0.217971 |
| Apathy/Indifference | −0.12 | −0.25 | 0.929020 |
| Disinhibition | −0.11 | −0.53 | 0.336992 |
| Irritabililty/Lability | 0.15 | 0.04 | 0.574638 |
| Aberr. Mot. Behavior | 0.15 | 0.01 | 0.945476 |
| Night-time Behavior | 0.46 | 0.05 | 0.586877 |
| App./Eating Change | 0.02 | 0.00 | 0.590741 |

ITT—intend to treat study population
OC—observed cases

Discussion

This study provides evidence that NMDA receptor modulation to reduce glutamate-induced excitotoxicity alleviates behavioral symptoms in AD. This novel neurochemical approach is distinct from the cholinomimetic mechanism of all currently approved treatments for AD and the behavioral disorders associated therewith.

Responder analyses (rates of individual responders) are often performed to assess the clinical relevance of results. In the present study, a significant difference in the predefined multi-endpoint responder criterion was observed. The treatment effects seen in the areas of cognition and function seemed to translate into behavioral (less agitation in the AE reports) and caregiver (less hours spent assisting) mitigation.

### Therapy Example 2

### Effect of Memantine Administered in Combination with Donepezil on Behavioral Outcomes in Patients Suffering from Moderate to Severe Alzheimer's Disease

A 24 week, double-blind, placebo-controlled trial was conducted in moderate to severe Alzheimer's patients (N=404) treated with ongoing donepezil therapy and randomized to memantine or placebo. Behavioral symptoms were assessed using the NPI administered at baseline, Week 12 and the final visit (Week 24). The trial already had established benefits of memantine on functional, cognitive, and global measures. The statistical analysis was based on the ITT population using the last observation carried forward (LOCF) and observed case (OC) approaches.

Therapeutic benefits of combining memantine with a stable dose of a commonly used AChEI in patients with moderate to severe Alzheimer's disease (mean MMSE of 10 at entry) were observed on behavioral (NPI) measures. Of the 12 single item behavioral domains measured by NPI, memantine treatment resulted in significant improvement in agitation/aggression, irritability/lability, and appetite/eating change compared to placebo.

Methods

Participants

This study enrolled four hundred and four patients diagnosed with probable AD according to the National Institute of Neurological and Communicative Disorders and Stroke—Alzheimer's Disease and Related Disorders Association (NINCDS-ADRDA) criteria with moderate to severe stages of illness. Inclusion criteria were as follows: Mini-Mental State Examination (MMSE) score of 5-14 at screening and at baseline; minimum age of 50 years; a recent MRI or CT scan consistent with a diagnosis of probable AD; on AChEI therapy, which was defined by the protocol to be donepezil, for more than 6 months prior to entrance into the trial and at a stable dose (5-10 mg/day) for at least 3 months prior to entrance in the trial; a knowledgeable and reliable caregiver to accompany the patient to research visits and oversee the administration of the investigational agent during the trial; residence in the community; ambulatory or ambulatory-aided (i.e. walker or cane) ability; and stable medical condition.

Patients were excluded for clinically significant reasons; B12 or folate deficiency; active pulmonary, gastrointestinal, renal, hepatic, endocrine, or cardiovascular disease; other psychiatric or central nervous system disorders; CT or MRI evidence of clinically significant central nervous system disorders other than probable AD; dementia complicated by other organic disease; or a modified Hachinski Ischemia Score30 greater than 4 at screening.

Interventions

This was a prospective, randomized, placebo-controlled, parallel-arm, fixed-dose trial in which participants were assigned to double-blind treatment for 24 weeks with a 1-2 week single-blind, placebo lead-in phase prior to randomization. The initial dose of memantine was 5 mg/day with escalation to 20 mg/day over the first 3 weeks of double-blind treatment. All patients were to maintain their protocol-defined AChEI therapy at entry dose for the duration of the study. From week 3 to the end of week 8 of double-blind treatment, transient dosage adjustments for memantine treatment were permitted for patients experiencing dose-limiting adverse events. All memantine-treated patients were required to receive the target dose of 20 mg/day by the end of week 8, and patients not tolerating the target dose at that time were discontinued.

Patients were randomly allocated in permuted blocks of four to one of the two treatment. Blinded study medication was supplied to each study site for dispensation in blister packs at each visit. No unblinding occurred during the trial. Compliance monitoring was conducted via pill counts, and over 95% of both treatment groups had >75% compliance (95% for the placebo/AChEI treatment group and 96.5% for the memantine/AChEI treatment group).

Objectives

The primary objective was to compare the efficacy of memantine to placebo in patients with moderate to severe dementia of the Alzheimer's type receiving stable doses of AChEI. The secondary objectives were to examine other measures of efficacy and to evaluate the safety and tolerability of memantine in these patients.

Outcomes

Cognitive, functional, global, and behavioral outcome measures were obtained at screening, baseline, and at the end of weeks 4, 8, 12, 18, and 24. Participants who discontinued prematurely were seen for a final evaluation. The efficacy parameters were the change from baseline on the SIB, a modified ADCS-ADL Inventory score at week 24, and the NPI. The 12-item version of the NPI was used here, with a total score ranging from 0-144. Higher scores reflect greater symptomatology. The NPI was assessed at baseline, at the end of week 12, and at the final visit.

Sample Size

Assuming an effect size of 0.35, a sample size of at least 170 patients in each treatment group provided a 90% power at an alpha level of 0.05 (two-sided), based on a two-sample t-test for change from baseline to Week 24 in both SIB and ADCS-ADL Inventory scores.

Statistical Methods

Three populations were considered in the statistical analyses. The randomized population consisted of all participants randomized into the study (n=404); the safety population consisted of all randomized participants who received at least one dose of double-blind study medication (n=403); the Intent-To-Treat (ITT) population consisted of participants in the safety population who completed at least one post-baseline SIB or ADCS-ADL assessment (n=395). All efficacy analyses were based on the ITT population. Primary efficacy analyses were conducted using the Last Observation Carried Forward (LOCF) approach for missing data imputation. Supportive analyses were performed using the Observed Case (OC) approach.

Results

Participants

Of the 404 patients who entered the study, 201 were assigned placebo/AChEI and 203 were assigned memantine/AChEI. More participants in the memantine/AChEI group (n=172; 85.1%) completed the study than in the placebo/AChEI group (n=150; 74.6%; p=0.011). The demographic and clinical characteristics of the two groups at baseline are summarized in Table 2.

TABLE 2
Demographic and Clinical Characteristics at Baseline

| | Placebo/AChEI | Memantine/AChEI |
|---|---|---|
| Safety Population | (n = 201) | (n = 202) |
| Male/Female | 67/134 | 74/128 |
| Mean age, yrs (SD) | 75.5 (8.73) | 75.5 (8.45) |
| Mean weight, lbs (SD) | 146.0 (31.07) | 155.5 (31.49)** |
| Race-Caucasian, n (%) | 186 (92.5) | 182 (90.1) |
| Other active medical | 200 (99.5) | 201 (99.5) |
| conditions, n (%) | | |
| Mean MMSE score (SD) | 10.2 (2.98) | 9.9 (3.13) |
| | Placebo/AChEI | Memantine/AChEI |
| Intent to Treat Population | (n = 197) | (n = 198) |
| Mean SIB score (SD) | 79.8 (14.18) | 77.8 (15.46) |
| Mean ADCS-ADL score (SD) | 36.2 (9.32) | 35.9 (9.75) |
| Mean NPI score (SD) | 13.8 (12.83) | 13.7 (14.11) |
| Mean BGP Care Dependency | 9.2 (5.99) | 8.9 (5.83) |
| score (SD) | | |
| Mean duration of AChEI | 129 (70.3) | 126 (64.9) |
| treatment, weeks (SD) | | |
| Mean dose AChEI^{†}, mg (SD) | 9.49 (1.88) | 9.25 (1.79) |

**p < .01 vs. placebo/AChEI
^{†}Donepezil
MMSE = Mini-Mental State Examination
SIB = Severe Impairment Battery
ADCS-ADL = Alzheimer's Disease Cooperative Study - Activities of Daily Living Inventory
NPI = Neuropsychiatric Inventory
BGP = Behavioral Rating Scale for Geriatric Patients

Efficacy

Statistically significant benefits of combined treatment with memantine/AChEI over treatment with placebo/AChEI were observed on all outcome measures as presented below. Table 3 summarizes efficacy outcomes at week 24 and at endpoint, presenting results for the ITT population using both the OC and LOCF approaches.

TABLE 3
Efficacy Outcomes at Week 24 (OC) and at Endpoint (LOCF)

| Assessment: | | | | |
|---|---|---|---|---|
| Least Squares | | | | |
| Mean Change | PLACEBO/ACHEI | | MEMANTINE/ACHEI | |
| from Baseline | LOCF (SE) (n) | OC (SE) (n) | LOCF (SE) (n) | OC (SE) (n) |
| SIB | −2.5 (0.69) (196) | −2.4 (0.74) (153) | 0.9 (0.67) (198)*** | 1.0 (0.70) (171)*** |
| ADCS-ADL | −3.4 (0.51) (197) | −3.3 (0.55) (152) | −2.0 (0.50) (198)* | −1.7 (0.51) (172)* |
| CIBIC-Plus† | 4.66 (0.075) (196) | 4.64 (0.087) (152) | 4.41 (0.074) (198)* | 4.38 (0.081) (172)* |
| NPI | 3.7 (0.99) (189) | 2.9 (1.06) (152) | −0.1 (0.98) (193)** | −0.5 (0.99) (171)** |
| BGP Care | 2.3 (0.38) (179) | 2.2 (0.40) (151) | 0.8 (0.37) (185)*** | 0.6 (0.37) (172)*** |
| Dependency | | | | |
| subscale | | | | |

*p < .05;
**p < .01;
***p < .001 vs. placebo/AChEI
†arithmetic mean
ITT = Intent-to-Treat population
SE = Standard Error
OC = Observed Case approach
LOCF = Last Observation Carried Forward approach
SIB = Severe Impairment Battery
ADCS-ADL = Alzheimer's Disease Cooperative Study - Activities of Daily Living Inventory
CIBIC-Plus = Clinician's Interview-Based Impression of Change plus Caregiver Input
NPI = Neuropsychiatric Inventory
BGP = Behavioral Rating Scale for Geriatric Patients

Analyses using the LOCF approach showed a statistically significant benefit of memantine/AChEI over treatment with placebo/AChEI on the SIB (p<0.001) and the ADCS-ADL (p=0.028), as did analyses using the OC approach (p<0.001 for SIB, p=0.020 for ADCS-ADL).

The total NPI score was significantly lower for the memantine/AChEI group compared to the placebo/AChEI group, at week 24 (p<0.01 using both OC and LOCF) representing fewer behavioral disturbances and psychiatric symptoms for patients in the memantine/AChEI group. The mean change in total NPI scores over time and demonstrates that the difference between the two groups was statistically significant at week 12 (p<0.001 using OC) and at week 24 (p=0.01 using OC) as well as at endpoint (p=0.002 using LOCF).

Behavioural symptoms were assessed using the NPI administered at baseline at week 24. A statistically significant treatment difference was observed as a reduction in behavioural disturbances and psychiatric symptoms in memantine-treated patients and a worsening in placebo-treated patients. The NPI domains demonstrating statistically significant treatment differences at week 24 were agitation/aggression (p<0.001) and irritability/lability (p=0.003). See Table 4.″.

TABLE 4
Change (baseline to week 24) in NPI by domain in
patients receiving memantine and donepezil
NPI by domain, ITT, OC

| | Placebo/ | Memantine/ | p-value for |
|---|---|---|---|
| DOMAIN | AChEI | AChEI | change |
| Delusions | 0.3 | 0.1 | 0.321 |
| Hallucinations | 0.1 | 0.1 | 0.804 |
| Agitation/Aggression | 0.7 | −0.2 | ≦0.001 |
| Depression/Dysphoria | 0.0 | 0.1 | 0.866 |
| Anxiety | 0.0 | −0.1 | 0.661 |
| Elation/Euphoria | 0.0 | 0.0 | 0.610 |
| Apathy/Indifference | 0.2 | −0.4 | 0.090 |
| Disinhibition | 0.1 | 0.0 | 0.499 |
| Irritabililty/Lability | 0.6 | −0.2 | 0.003 |
| Aberr. Mot. Behavior | 0.2 | 0.3 | 0.884 |
| Night-time Behavior | 0.4 | 0.0 | 0.073 |
| App./Eating Change | 0.1 | −0.3 | 0.159 |

ITT—intend to treat study population
OC—observed cases

Discussion

This was the first prospective, double-blind, placebo-controlled study examining the benefits of the dual therapy of an NMDA receptor antagonist in AD patients on a stable dose of a protocol-defined AChEI in AD patients. Treatment with memantine/AChEI was superior to treatment with placebo/AChEI in patients with moderate to severe AD residing in the community. Measures of cognitive function, activities of daily living, behavior, and clinical global status were all significantly improved with the combination of memantine and AChEI compared to placebo/AChEI. Treatment with memantine/AChEI resulted in improved cognitive function versus baseline values, whereas treatment with placebo/AChEI was associated with progressive cognitive decline over the 6-month trial.

Conclusion

The results from this trial confirm the safety and efficacy of memantine in the treatment of behavioral disorders in patients with moderate to severe AD and demonstrate that treatment with memantine concomitantly with the AChEI is superior to treatment with the AChEI alone in these patients.

### Therapy Example 3

This example presents results from the five (5) clinical trials two of which are described above in Example 1 (MZ-9065) and Example 2 (MD-02). In addition, results from two additional trials, MD-01, MD-10 and MD-12, are presented.

MD-01: MD-01 was a trial evaluating memantine as monotherapy for the treatment of patients with moderate-to-severe AD lasting for 24 weeks. About 350 patients were enrolled. Efficacy was evaluated using the Severe Impairment Battery (SIB), the Assessment of Daily Living (ADL) and the CIBIC-plus Scales.

MD-10: MD-10 was a randomized, double-blind, placebo-controlled evaluation of the safety and efficacy of memantine in patients with mild to moderate dementia of the Alzheimer's type. The primary endpoints for MD-10 were ADAS-cog and CIBIC-plus. Other endpoints for MD-10 included ADCS-ADL, NPI, and RUD (Resource Utilization in Dementia).

MD-12: MD-12 was a randomized, double-blind, placebo-controlled evaluation of the safety and efficacy of memantine in patients with mild to moderate dementia of the Alzheimer's type who had been on stable chronic doses of a cholinesterase inhibitors (donepezil, rivastigmine, or galantamine). The primary endpoints for MD-12 were ADAS-cog and CIBIC-plus. Other endpoints for MD-12 included ADCS-ADL23, NPI, MMSE and RUD (Resource Utilization in Dementia).

The five memantine trials were evaluated using an analysis of patient for a population having an NPI agitation subscale score of >4 (less agitated) and for a population having an NPI score of <4 (more agitated). For each NPI population, changes in SIB, ADAS-cog and/or ADL were calculated using the least square (LS) mean difference for memantine and placebo-treated patients. Similarly, for each NPI population, the raw mean difference between memantine and placebo was calculated for the CIBIC-plus results.

For primary efficacy measurements the comparison between memantine and placebo was performed using two-way analysis of covariance (ANCOVA) with treatment group and center as the two factors, and the baseline scores as covariate. Both the LOCF imputation approach and the OC approach are performed at the end of the trials.

For secondary efficacy measurements, the comparison between memantine and placebo was performed using two-way analysis of covariance (ANCOVA) with treatment group and center as the two main effects, and the baseline scores as covariate. Descriptive statistics were calculated by Visit. Again, all analyses were performed using both the LOCF imputation approach and the OC approach.

Results of ANCOVA were summarized using least square (LS) means for each treatment group with corresponding standard error (SE), the between-treatment (2-24 week) difference in the least square means with corresponding 95% confidence interval, and the between-treatment p-value corresponds to the SAS Type III sum of squares.

In data Tables 5-19, the Overall Memantine versus Placebo columns of studies MD-01, MD-02, MD-10 and MD-12 used an ANCOVA model with baseline total score as covariate, treatment and study center as factors for SIB, ADL or ADAS-cog; CMH test controlling for study center for CIBIC-Plus. For MZ-9605, results are based on arithmetic means and the Wilcoxon-Mann-Whitney test. The Agitation score columns/NPI/antipsychotics columns, except CIBIS-Plus, calculated the p-values and LS mean differences within the subgroups from an ANCOVA model with baseline total score as covariate, subgroup (yes/no), treatment, and treatment by subgroup interactions as factors. For CIBIS-Plus, the p-values and raw mean differences within a subgroup were calculated using the CMH test controlling for study center.

Results

MD-02: For MD-02, the number of patients having an agitation score of greater than or equal to 4 (more agitated) was 60, with 30 receiving placebo and 30 receiving memantine. The number of patients having an agitation score of less than 4 was 334, with approximately half receiving memantine and half receiving placebo.

SIB (MD-02). The treatment effect as determined by the difference between memantine and placebo between the two NPI populations was measured. The memantine-treated groups demonstrated improvement over the placebo-treated groups in both NPI≧4, and especially NPI<4, (LS mean 3.8, p=0.0834; and LS mean 3.6, p=0.0003, respectively), the LS mean overall of the groups was 3.6. The combined populations showed significant improvement with memantine over placebo (p<0.001). Results are presented in Table 5.

ADCS-ADL (MD-02) The change in ADCS-ADL reflected improvement of both NPI populations with memantine (NPI≧4−LS mean 2.1, p=0.2065, NPI<4−LS mean 1.2, p=0.0790), with the difference between the two groups showing a greater difference, i.e., there was more improvement with memantine in the NPI≧4 sub-group. The LS mean overall of the groups was 1.40. The overall difference in response between the memantine-treated and placebo groups in the combined population is significant (p=0.028). See Table 5.

CIBIC-plus (MD-02) The change in CIBIC-plus reflected a significant improvement in response for the sub-group with NPI<4 with memantine v. placebo (raw mean −0.3, p=0.0311), as well as improvement in the NPI24 population (raw mean difference −0.1, p=0.6875), with the overall response of the two NPI groups being significant (p=0.029) in favor of memantine over placebo (raw mean difference −0.27). See Table 5.

MD-01: For MD-01, the number of patients having an NPI score of greater than or equal to 4 (more agitated) was 68, with 34 receiving placebo and 34 receiving memantine. The number of patients having an NPI score of less than 4 was about 267, with approximately half receiving memantine and half receiving placebo.

SIB (MD-01) Similar to MD-02, the differences in SIB between the memantine and placebo-treated populations was significantly more pronounced in the more agitated NPI≧4 sub-group (LS mean 4.65, p=0.0602), than in the less impaired NPI<4 sub-group (LS mean −0.15, p=0.9017), and for the combined sub-populations was also improved (LS mean 0.60, p=0.616). Results are presented in Table 6.

ADCS-ADL (MD-01) The change in ADL scores sub-group with NPI≧4 was LS mean 1.18 (p=0.4286), with the memantine-treated group having a higher score (i.e., greater improvement, −3.2 v. −2.2 with placebo). However, the NPI≧4 group had a greater LS mean difference, e.g., improvement, with memantine over placebo than did the NPI<4 (LS mean 0.36, p=0.6299). Overall, the combined groups demonstrated an LS mean of 0.70 (p=0.282). See Table 6.

CIBIC-plus (MD-01) There was no raw mean difference between memantine and placebo in the NPI≧4 population (raw mean 0, p=0.1764), and a slight difference (improvement) observed in the NPI<4 sub-group (raw mean difference −0.2, p=0.3094). The combined populations showed improvement with a trend toward significance (raw mean difference −0.30, p=0.182). See Table 6.

MD-12: For MD-12, the number of patients having an NPI score of greater than 4 (more agitated) was about 45, with half receiving placebo and half receiving memantine. The number of patients having an NPI score of less than 4 was about 380, with approximately half receiving memantine and half receiving placebo.

ADAS-cog (MD-12) For this study, the ADAS-cog replaces the SIB as the cognitive endpoint. Scores range from 0 to 70 with lower scores indicating lesser severity and a score of 70 representing the worst cognitive impairment. The difference between the LS mean in the two subgroups is −0.27 in the NPI≧4 subgroup and −0.72 in the NPI<4 subgroup. Overall, the difference is −0.70. See Table 7.

ADCS-ADL(MD-12). The LS mean in the NPI≧4 sub-population (3.54) is significantly higher than the LS mean for the NPI<4 sub-population (−0.56). See Table 7.

CIBIC-plus (MD-12). There was no difference between memantine and placebo in the NPI≧4 group, and a very slight difference (LS mean −0.1) in the NPI<4 subgroup. See Table 7.

MZ-9605: For MZ-9605, the number of patients having an agitation score of greater than 4 (more agitated) was about 54, with half receiving placebo and half receiving memantine. The number of patients having an NPI score of less than 4 was about 200, with approximately half receiving memantine and half receiving placebo.

SIB (MZ-9605) This trial demonstrated a significant improvement in the SIB in the NPI≧4 sub-group (LS mean 14.05, p=0.0001) between the memantine and placebo-treated patients. Similarly, a significant improvement was observed in the NPI<4 sub-group (LS mean 3.75, p=0.03250), but not to the extent observed in the “more agitated” NPI≧4 sub-group, which latter group improved from −14.4 with placebo to −0.5 with memantine, while the NPI<4 sub-group improved from −8.5 with placebo to −4.8 with memantine. Overall, the improvement between the patients treated with memantine was highly significant, mean 5.91 (p=0.0003). See Table 8.

ADCS-ADL (MZ-9605) Similar to the SIB, the LS mean in the more agitated population was significant, demonstrating improvement with memantine (LS mean 4.97, p=0.0050). There was also improvement observed in the NPI<4 sub-group (LS mean 1.18, p=0.1983). The NPI≧4 subgroup had an overall higher score (i.e., improvement), −1.2 (up from −6.2), when administered memantine compared with the NPI sub-group, whose score improved to −3.5 (up from −4.8). The overall combined improvement was significant (LS mean 2.06, p=0.0217), with an improvement from −5.08 to −3.02. See Table 8.

CIBIC-plus (MZ-9605) The raw mean difference between the two NPI sub-groups showed improvement with memantine. The NPI≧4 subgroup showed a decreased score from 4.9 with placebo to 4.4 (e.g., improvement) in the CIBIC-plus (raw mean −0.5, p=0.8330), and the NPI<4 sub-group exhibited a decrease from 4.7 with placebo to 4.5 with memantine (raw mean −0.2, p=0.3350). The combined groups exhibited an overall decrease from 4.73 to 4.48 with memantine (raw mean difference −0.25), with a trend approaching significance (p=0.0644). See Table 8.

MD-10: For MD-10, the number of patients having an NPI score of greater than 4 (more agitated) was about 44, with half receiving placebo and half receiving memantine. The number of patients having an NPI score of less than 4 was about 349, with approximately half receiving memantine and half receiving placebo.

ADAS-cog (MD-10) For this study, the ADAS-cog replaces the SIB as the cognitive endpoint. The difference between the LS mean in the two subgroups is −1.39 in the NPI≧4 subgroup and −1.87 in the NPI<4 subgroup. Overall, the LS mean difference of −1.90 is significant (p=0.003). See Table 9.

ADCS-ADL (MD-10). The ADAS-ADL showed a difference between the LS in the two subgroup as −2.66 in the NPI≧4 sub-population and 0.65 in the NPI<4 sub-population. Overall, the difference was 0.10. See Table 9.

CIBIC-plus (MD-10). There was no difference between memantine and placebo in the NPI≧4 group, but a difference (LS mean −0.36) in the NPI<4 subgroup. See Table 9.

The above results show an increased mean difference, or a more pronounced improvement, in cognitive and functional endpoints in moderate to severe AD patients having a higher NPI agitation sub-scale score. Increased mean differences in the functional endpoint was also observed in more impaired patients with mild to moderate AD (ADAS-ADL, MD-12). Taken together, these data demonstrate that memantine is effective for mitigating agitation in patients with AD.

### Therapy Example 4

Patients in the same five trials described above were stratified into two sub-groups in a second analysis, those with an NPI total score in the top quartile (≧75%), or those more severely impaired at baseline, and those with an NPI total score <75%, or less severe impairment at baseline. Similar to as in Example 1, results were evaluated using the change in SIB (cognitive endpoint), and change in ADCS-ADL (functional endpoint), using the LS mean. The change in the CIBIC-plus (global endpoint) was assessed using the raw mean.

MD-02: For MD-02, the number of patients having an NPI score of greater than 75% (more impaired) was 102, with 52 receiving placebo and 50 receiving memantine. The number of patients having an NPI score in the <75% quartile was 292, with approximately 148 receiving memantine and 144 receiving placebo.

SIB (MD-02). The treatment effect was determined by the difference between memantine and placebo between the two NPI populations. The memantine-treated groups demonstrated improvement over the placebo-treated groups in both NPI≧75%, and especially NPI<75% (LS mean 6.1, p=0.0003; and LS mean 2.5, p=0.0108, respectively). The LS mean overall of the combined populations was significant at −3.40 (p<0.001). Results are presented in Table 10.

ADCS-ADL (MD-02). There also was improvement of both NPI populations with memantine (NPI≧75%−LS mean 1.73, p=0.1642, NPI<75%−LS mean 1.17, p=0.1089), with the greater difference being in the more impaired NPI≧75% population. The overall difference in response between the memantine treated and placebo groups in the combined population was significant (p=0.028), with an LS mean of −1.40. See Table 10.

CIBIC-plus (MD-02). The change in CIBIC-plus reflected an improvement in response for the sub-group with NPI≧75% with memantine v. placebo (raw mean −0.3, p=0.4857), as well as improvement in the NPI<75% population (raw mean difference −0.2, p=0.0943), with the overall response of the two NPI groups being significant (p=0.027) in favor of memantine over placebo (raw mean difference −0.25). See Table 10.

MD-01: For MD-01, the number of patients having an NPI score in the top quartile (≧75%-more impaired) was 90, with 35 receiving placebo and 55 receiving memantine. The number of patients having an NPI score falling in the <75% quartile was about 245, with approximately 115 or 116 receiving memantine and 130 receiving placebo.

SIB (MD-01). Similar to MD-02, the differences in SIB between the memantine and placebo-treated populations was more pronounced in the more agitated NPI≧75% sub-group (LS mean 3.5, p=0.1073), than in the less agitated NPI<75% sub-group (LS mean 0.3, p=0.7998). The combined LS mean difference for both sub-populations was 0.60 (p=0.616). Results are presented in Table 11.

ADCS-ADL (MD-01). The change in ADL scores sub-group with NPI≧75% was LS mean −0.05 (p=0.9685). The change in the NPI<75% population was LS mean 0.87 (p=0.2652). Overall, the combined groups demonstrated an LS mean of 0.70 (p=0.282). See Table 11.

CIBIC-plus (MS-01). There was a raw mean difference between memantine and placebo in the NPI>75% population (raw mean −0.2, p=0.3486) favoring memantine, and similarly, an improvement observed in the NPI<75% sub-group (raw mean difference −0.2, p=0.2040). The combined populations showed a difference approaching significance (raw mean −0.30, p=0.182) in favor of memantine. See Table 11.

MZ-9605: For MZ-9605, the number of patients having an agitation score of greater than 75% (more impaired) was about 66, with 31 receiving placebo and 35 receiving memantine. The number of patients having an agitation score of less than 75% was about 186, with approximately 91 receiving memantine and 95 receiving placebo.

SIB (MZ-9605). This trial demonstrated a significant improvement in the SIB in the NPI≧75% sub-group (LS mean 13.3, p=0.0001) between the memantine and placebo-treated patients. Similarly, an improvement with memantine was observed in the NPI<75% sub-group (LS mean 3.36, p=0.0630). Overall, the improvement between the patients in both groups treated with memantine was highly significant, mean 5.91 (p=0.0003). See Table 12.

ADCS-ADL (MZ-9605). Similar to with the SIB, the LS mean in the more impaired NPI≧75% population was significant, demonstrating improvement with memantine (LS mean −3.47, p=0.0305). Similar to the SIB, there was observed improvement observed in the NPI<75% sub-group (as shown by a lower score, LS mean 1.48, p=0.1200). The overall combined improvement was significant (LS mean 2.06, p=0.0217), with an improvement from −5.08 with placebo to −3.02 with memantine. See Table 12.

For ADCS-ADL, a positive change from baseline signals improvement with memantine. Results for MD-02 and MZ-9605 are very consistent, with improvements with memantine, especially in MZ-9605. MD-01 also shows a trend towards improvement with memantine.

CIBIC-plus (MZ-9605). There was a raw mean difference between memantine and placebo in the NPI≧75% population (raw mean −0.4, p=0.4720) favoring memantine, and similarly, an improvement observed in the NPI<75% sub-group (raw mean difference −0.2, p=0.2510). The combined populations showed a difference approaching significance (raw mean −0.25, p=0.0644) in favor of memantine. See Table 12.

For CIBIC-plus, a difference between memantine and placebo less than zero, i.e., a negative number, indicates memantine is better than placebo. The general trend across the studies shows MD-9605 with the most significant result favoring memantine, followed by MD-02, then MD-01. The variation is more pronounced in the top 25% quartile (NPI≧75%).

MD-12: For MD-12, the number of patients in the top 25% quartile (NPI≧75%) was 110, with approximately half receiving memantine and half receiving placebo. For patients in the NPI<75% group, there were about 317 patients, with about half receiving memantine and half receiving placebo.

ADAS-cog (MD-12). There was improvement with memantine v. placebo in the top 25% quartile (NPI≧75%), with an LS mean of −1.53 v. a change of −0.39 in the NPI<75% group. This change was greater than the overall change of −0.70. See Table 13.

ADCS-ADL (MD-12). There was significant improvement in the NPI≧75% group (more severe at baseline), with an LS mean of 2.0. The placebo did better than memantine in the NPI<75% group, with a change of −0.76. Overall, there was improvement with memantine, with a positive LS mean of −0.20. See Table 13.

CIBIC-plus (MD-12). There was no difference in MD-12 in patients in the NPI≧75% receiving either placebo or memantine (LS mean 0.0). There was only a slight change (improvement) with memantine in the NPI<75% population (LS mean −0.041). Overall, there was a change favoring memantine of −0.04. See Table 13.

MD-10: For MD-10, the number of patients in the top 25% quartile (NPI≧75%) was 98, with approximately half receiving memantine and half receiving placebo. For patients in the NPI<75% group, there were about 295 patients, with about half receiving memantine and half receiving placebo.

ADAS-cog (MD-10). There was improvement with memantine versus placebo in the top 25% quartile (NPI≧75%), with an LS mean of −2.53 versus a change of −1.59 in the NPI<75% group. The overall change was −1.90. See Table 14.

ADCS-ADL (MD-10). There was improvement in the NPI≧75% group (more severe at baseline), with an LS mean difference of 0.70. Additionally, there was improvement in the NPI<75% group, with an LS mean difference of 0.11. Overall, there was improvement with memantine, with a positive LS mean of 0.10. See Table 14.

CIBIC-plus (MD-10). There was improvement in the NPI≧75% group, with a LS mean change of −0.3. Additionally, there was an improvement in the NPI<75% population, with a LS mean change of −0.32. Overall, there was a change favoring memantine of −0.30. See Table 14.

### Therapy Example 5

A sub-population of patients from the five trials, MD-01, MD-02, MD-10, MD-12, and MZ-9605, were concurrently taking antipsychotics. An analysis of results is presented in Tables 15-19. Results demonstrate that memantine improves cognitive (SIB), functional (ADCS-ADL) and CIBIC-Plus endpoints in MD-02. In MZ-9605, memantine showed improvement in both the cognitive (SIB) and functional (ADCS-ADL) endpoints. Finally, in MD-10, memantine showed improvement in both the ADAS-cog and CIBIC-Plus endpoints.

TABLE 5
BASELINE NPI AGITATION SCORE (>4 v. <4) ADJUSTED AT LAST WEEK (ITT, LOCF)

| | | | AGITATION | | | AGITATION | | | OVERALL MEM v. | | |
|---|---|---|---|---|---|---|---|---|---|---|---|
| | | TREATMENT | ≧=4 | | | <4 | | | PLACEBO | | |
| STUDY | PARAMETER | GROUP | N = y | mean | p-value | N = n | mean | p-value | n | LS mean | p-value |
| MD-02 | SIB (change) | MEM | 30 | 1.1 | 0.0834 | 168 | 1.0 | 0.0003 | 198 | 0.90 | <0.001 |
| WEEK 24 | | PLA | 30 | −2.6 | | 166 | −2.2 | | 196 | −2.50 | |
| | | LS Mean Diff. | 60 | 3.8 | | 334 | 3.4 | | 394 | −3.40 | |
| | ADCS-ADL | MEM | 30 | −2.0 | 0.2065 | 168 | −1.8 | 0.790 | 198 | −2.00 | 0.028 |
| | (change) | PLA | 30 | −4.1 | | 167 | −3.0 | | 197 | −3.40 | |
| | | LS Mean Diff. | 60 | 2.1 | | 335 | 1.2 | | 395 | −1.40 | |
| | CIBIC-PLUS | MEM | 30 | −4.5 | 0.6875 | 168 | 4.4 | 0.0311 | 198 | 4.41 | 0.027 |
| | | PLA | 30 | 4.7 | | 166 | 4.7 | | 196 | 4.66 | |
| | | Raw Mean Diff. | 60 | −0.1 | | 334 | −0.3 | | 394 | −0.25 | |

Tables

TABLE 6
BASELINE NPI AGITATION SCORE (>4 v. <4) ADJUSTED AT LAST WEEK (ITT, LOCF)

| | | | AGITATION | | | AGITATION | | | OVERALL MEM v. | | |
|---|---|---|---|---|---|---|---|---|---|---|---|
| | | TREATMENT | ≧=4 | | | <4 | | | PLACEBO | | |
| STUDY | PARAMETER | GROUP | N = y | mean | p-value | N = n | mean | p-value | n | LS mean | p-value |
| MD-01 | SIB (change) | MEM | 34 | −1.0 | 0.0602 | 136 | −1.9 | 0.9017 | 170 | −2.00 | 0.616 |
| WEEK 24 | | PLA | 34 | −6.00 | | 131 | −1.8 | | 165 | −2.50 | |
| | | LS Mean Diff. | 68 | 4.65 | | 267 | −0.15 | | 335 | 0.60 | |
| | ADCS-ADL | MEM | 34 | −2.2 | 0.4286 | 168 | −1.3 | 0.6299 | 171 | −2.00 | 0.282 |
| | (change) | PLA | 34 | −3.2 | | 167 | −1.8 | | 165 | −2.70 | |
| | | LS Mean Diff. | 68 | 1.18 | | 335 | 0.36 | | 336 | 0.70 | |
| | CIBIC-PLUS | MEM | 34 | 4.7 | 0.1764 | 137 | 4.3 | 0.3094 | 171 | 4.30 | 0.182 |
| | | PLA | 33 | 4.7 | | 130 | 4.5 | | 163 | 4.60 | |
| | | Raw Mean Diff. | 67 | 0.0 | | 267 | −0.2 | | 334 | −0.30 | |

TABLE 7
BASELINE NPI AGITATION SCORE (>4 v. <4) ADJUSTED AT LAST WEEK (ITT, LOCF)

| | | | AGITATION | | | AGITATION | | | OVERALL MEM v. | | |
|---|---|---|---|---|---|---|---|---|---|---|---|
| | | TREATMENT | ≧=4 | | | <4 | | | PLACEBO | | |
| STUDY | PARAMETER | GROUP | N = y | mean | p-value | N = n | mean | p-value | n | LS mean | p-value |
| MD-12 | ADAS-cog | MEM | 20 | 0.98 | 0.8791 | 192 | 0.35 | 0.2424 | 212 | 0.10 | 0.184 |
| WEEK 24 | (change) | PLA | 25 | 1.19 | | 187 | 1.04 | | 212 | 0.80 | |
| | | LS Mean Diff. | 45 | −0.27 | | 379 | −0.72 | | 424 | −0.70 | |
| | ADCS-ADL | MEM | 20 | −3.7 | 0.1242 | 194 | −2.8 | 0.4722 | 214 | −3.00 | 0.816 |
| | (change) | PLA | 25 | −7.2 | | 188 | −2.3 | | 213 | −2.90 | |
| | | LS Mean Diff. | 45 | 3.54 | | 382 | −0.56 | | 427 | −0.20 | |
| | CIBIC-PLUS | MEM | 20 | 4.7 | 0.9050 | 194 | 4.3 | 0.7400 | 214 | 4.38 | 0.843 |
| | | PLA | 25 | 4.7 | | 188 | 4.4 | | 213 | 4.42 | |
| | | Raw Mean Diff. | 45 | 0.0 | | 382 | −0.1 | | 427 | −0.04 | |

TABLE 8
BASELINE NPI AGITATION SCORE (>4 v. <4) ADJUSTED AT LAST WEEK (ITT, LOCF)

| | | | AGITATION | | | AGITATION | | | OVERALL MEM v. | | |
|---|---|---|---|---|---|---|---|---|---|---|---|
| | | TREATMENT | ≧=4 | | | <4 | | | PLACEBO | | |
| STUDY | PARAMETER | GROUP | N = y | mean | p-value | N = n | mean | p-value | n | LS mean | p-value |
| MZ-9605 | SIB (change) | MEM | 26 | −0.5 | 0.0001 | 100 | −4.8 | 0.0325 | 126 | −3.93 | 0.0003 |
| WEEK 28 | | PLA | 28 | −14.4 | | 98 | −8.5 | | 126 | −9.84 | |
| | | LS Mean Diff. | 54 | 14.05 | | 198 | 3.75 | | 252 | 5.91 | |
| | ADCS-ADL | MEM | 26 | −1.2 | 0.0050 | 100 | −3.5 | 0.1983 | 126 | −3.02 | 0.00217 |
| | (change) | PLA | 28 | −6.2 | | 98 | −4.8 | | 126 | −5.08 | |
| | | LS Mean Diff. | 54 | −4.97 | | 198 | 1.18 | | 252 | 2.06 | |
| | CIBIC-PLUS | MEM | 26 | 4.4 | 0.8330 | 100 | 4.5 | 0.3350 | 126 | 4.48 | 0.0644 |
| | | PLA | 28 | 4.9 | | 98 | 4.7 | | 126 | 4.73 | |
| | | Raw Mean Diff. | 54 | −0.5 | | 198 | −0.2 | | 252 | −0.25 | |

TABLE 9
BASELINE NPI AGITATION SCORE (>4 v. <4) ADJUSTED AT LAST WEEK (ITT, LOCF)

| | | | AGITATION | | | AGITATION | | | OVERALL MEM v. | | |
|---|---|---|---|---|---|---|---|---|---|---|---|
| | | TREATMENT | ≧=4 | | | <4 | | | PLACEBO | | |
| STUDY | PARAMETER | GROUP | N = y | mean | p-value | N = n | mean | p-value | n | LS mean | p-value |
| MD-10 | ADAS-cog | MEM | 22 | 0.86 | 0.4661 | 173 | −0.49 | 0.0060 | 195 | −0.80 | 0.003 |
| WEEK 24 | (change) | PLA | 22 | 2.23 | | 176 | 1.38 | | 198 | 1.10 | |
| | | LS Mean Diff. | 44 | −1.39 | | 349 | −1.87 | | 393 | −1.90 | |
| | ADCS-ADL | MEM | 22 | −5.05 | 0.3125 | 174 | −3 | 0.4846 | 196 | −2.90 | 0.890 |
| | (change) | PLA | 22 | −1.91 | | 176 | −3.8 | | 198 | −3.00 | |
| | | LS Mean Diff. | 44 | −2.66 | | 350 | 0.65 | | 394 | 0.10 | |
| | CIBIC-PLUS | MEM | 22 | 4.36 | 0.8702 | 174 | 4.18 | 0.0008 | 196 | 4.20 | 0.004 |
| | | PLA | 22 | 4.36 | | 175 | 4.54 | | 197 | 4.50 | |
| | | Raw Mean Diff. | 44 | 0.0 | | 349 | −0.36 | | 393 | −0.30 | |

TABLE 10
NPI total score (Top quartile v. others) adjusted analysis at last week (ITT, LOCF)

| | | | NPI | | | NPI | | | OVERALL MEM v. | | |
|---|---|---|---|---|---|---|---|---|---|---|---|
| | | TREATMENT | ≧=75% | | | <75% | | | PLACEBO | | |
| STUDY | PARAMETER | GROUP | N = y | mean | p-value | N = n | mean | p-value | n | LS mean | p-value |
| MZ-02 | SIB (change) | MEM | 50 | −1.4 | 0.0003 | 148 | 0.9 | 0.0108 | 198 | 0.90 | <0.001 |
| WEEK 24 | | PLA | 52 | −4.4 | | 144 | −1.5 | | 196 | −2.50 | |
| Q75 = 19 | | LS Mean Diff. | 102 | 6.1 | | 292 | 2.5 | | 394 | −3.40 | |
| | ADCS-ADL | MEM | 50 | −2.6 | 0.1642 | 100 | −1.6 | 0.0943 | 198 | −2.00 | 0.028 |
| | (change) | PLA | 52 | −4.3 | | 98 | −2.8 | | 197 | −3.40 | |
| | | LS Mean Diff. | 102 | −1.73 | | 198 | 1.17 | | 395 | −1.40 | |
| | CIBIC-PLUS | MEM | 50 | 4.4 | 0.4857 | 100 | 4.4 | 0.0943 | 198 | 4.41 | 0.027 |
| | | PLA | 52 | 4.8 | | 98 | 4.6 | | 196 | 4.66 | |
| | | Raw Mean Diff. | 102 | −0.3 | | 198 | −0.2 | | 394 | −0.25 | |

TABLE 11
NPI total score (Top quartile v. others) adjusted analysis at last week (ITT, LOCF)

| | | | NPI | | | NPI | | | OVERALL MEM v. | | |
|---|---|---|---|---|---|---|---|---|---|---|---|
| | | TREATMENT | ≧=75% | | | <75% | | | PLACEBO | | |
| STUDY | PARAMETER | GROUP | N = y | mean | p-value | N = n | mean | p-value | n | LS mean | p-value |
| MD-01 | SIB (change) | MEM | 55 | −2.8 | 0.1073 | 115 | −1.2 | 0.7998 | 170 | −2.00 | 0.616 |
| WEEK 24 | | PLA | 35 | −6.6 | | 130 | −1.5 | | 165 | −2.50 | |
| Q75 = 27 | | LS Mean Diff. | 90 | 3.5 | | 245 | 0.3 | | 335 | 0.60 | |
| | ADCS-ADL | MEM | 55 | −2.3 | 0.9685 | 116 | −1.1 | 0.2652 | 171 | −2.00 | 0.282 |
| | (change) | PLA | 35 | −2.2 | | 130 | −2.1 | | 165 | −2.70 | |
| | | LS Mean Diff. | 90 | −0.05 | | 246 | 0.87 | | 336 | 0.70 | |
| | CIBIC-PLUS | MEM | 54 | 4.5 | 0.3486 | 116 | 4.3 | 0.2040 | 171 | 4.30 | 0.182 |
| | | PLA | 34 | 4.7 | | 129 | 4.5 | | 163 | 4.60 | |
| | | Raw Mean Diff. | 89 | −0.2 | | 245 | −0.2 | | 334 | −0.30 | |

TABLE 12
Total score (Top quartile v. others) adjusted analysis at last week (ITT, LOCF)

| | | | NPI | | | NPI | | | OVERALL MEM v. | | |
|---|---|---|---|---|---|---|---|---|---|---|---|
| | | TREATMENT | ≧=75% | | | <75% | | | PLACEBO | | |
| STUDY | PARAMETER | GROUP | N = y | mean | p-value | N = n | mean | p-value | n | LS mean | p-value |
| MZ-9605 | SIB (change) | MEM | 35 | 0.03 | 0.0001 | 91 | −5.6 | 0.0630 | 126 | −3.93 | 0.0003 |
| WEEK 28 | | PLA | 31 | −12.5 | | 95 | −0.9 | | 126 | −9.84 | |
| Q75 = 28 | | LS Mean Diff. | 66 | 13.3 | | 186 | 3.36 | | 252 | 5.91 | |
| | ADCS-ADL | MEM | 35 | −2.1 | 0.0305 | 91 | −3.4 | 0.1200 | 126 | −3.02 | 0.0217 |
| | (change) | PLA | 31 | −5.7 | | 95 | −4.9 | | 126 | −5.08 | |
| | | LS Mean Diff. | 66 | −3.47 | | 186 | 1.48 | | 252 | 2.06 | |
| | CIBIC-PLUS | MEM | 35 | 4.6 | 0.4720 | 91 | 4.4 | 0.2510 | 126 | 4.48 | 0.0644 |
| | | PLA | 31 | 5.0 | | 95 | 4.7 | | 126 | 4.73 | |
| | | Raw Mean Diff. | 66 | −0.4 | | 186 | −0.2 | | 252 | −0.25 | |

TABLE 13
Total score (Top quartile v. others) adjusted analysis at last week (ITT, LOCF)

| | | | NPI | | | NPI | | | OVERALL MEM v. | | |
|---|---|---|---|---|---|---|---|---|---|---|---|
| | | TREATMENT | ≧=75% | | | <75% | | | PLACEBO | | |
| STUDY | PARAMETER | GROUP | N = y | mean | p-value | N = n | mean | p-value | n | LS mean | p-value |
| MD-12 | ADAS-cog | MEM | 54 | 0.05 | 0.1835 | 158 | 0.53 | 0.5628 | 212 | 0.10 | 0.184 |
| WEEK 24 | (change) | PLA | 54 | 1.55 | | 158 | 0.89 | | 212 | 0.80 | |
| | | LS Mean Diff. | 108 | −1.53 | | 316 | −0.39 | | 424 | −0.70 | |
| | ADCS-ADL | MEM | 56 | −2.8 | 0.1718 | 158 | −3.0 | 0.3791 | 214 | −3.00 | 0.816 |
| | (change) | PLA | 54 | −4.8 | | 159 | −2.2 | | 213 | −2.90 | |
| | | LS Mean Diff. | 110 | 2.00 | | 317 | −0.76 | | 427 | −0.20 | |
| | CIBIC-PLUS | MEM | 56 | 4.6 | 0.4833 | 158 | 4.3 | 0.6119 | 214 | 4.38 | 0.843 |
| | | PLA | 54 | 4.6 | | 159 | 4.4 | | 213 | 4.42 | |
| | | Raw Mean Diff. | 110 | 0.0 | | 317 | −0.1 | | 427 | −0.04 | |

TABLE 14
Total score (Top quartile v. others) adjusted analysis at last week (ITT, LOCF)

| | | | NPI | | | NPI | | | OVERALL MEM v. | | |
|---|---|---|---|---|---|---|---|---|---|---|---|
| | | TREATMENT | ≧=75% | | | <75% | | | PLACEBO | | |
| STUDY | PARAMETER | GROUP | N = y | mean | p-value | N = n | mean | p-value | n | LS mean | p-value |
| MD-10 | ADAS-cog | MEM | 46 | −1.18 | 0.0486 | 149 | −0.08 | 0.0313 | 195 | −0.80 | 0.003 |
| WEEK 24 | (change) | PLA | 52 | 1.37 | | 146 | 1.50 | | 198 | 1.10 | |
| | | LS Mean Diff. | 98 | −2.53 | | 295 | −1.59 | | 393 | −1.90 | |
| | ADCS-ADL | MEM | 46 | −5.04 | 0.6929 | 150 | −2.67 | 0.9146 | 196 | −2.90 | 0.890 |
| | (change) | PLA | 52 | −5.62 | | 146 | −2.86 | | 198 | −3.00 | |
| | | LS Mean Diff. | 98 | 0.70 | | 296 | 0.11 | | 394 | 0.10 | |
| | CIBIC-PLUS | MEM | 46 | 4.33 | 0.0930 | 150 | 4.16 | 0.0065 | 196 | 4.20 | 0.004 |
| | | PLA | 52 | 4.62 | | 145 | 4.48 | | 195 | 4.50 | |
| | | Raw Mean Diff. | 98 | −0.3 | | 295 | −0.32 | | 198 | −0.30 | |

TABLE 15
Antipsychotics usage adjusted analysis at last week (ITT, LOCF)

| | | | ANTIPSYCHOTICS | | | ANTIPSYCHOTICS | | | |
|---|---|---|---|---|---|---|---|---|---|
| | | TREATMENT | USAGE “YES” | | | USAGE “YES” | | | OVERALL MEM v. PLACEBO |
| STUDY | PARAMETER | GROUP | N = y | mean | p-value | N = n | mean | p-value | p-value |
| MD-02 | SIB (change) | MEM | 29 | 5.2 | 0.0001 | 169 | 0.3 | 0.0593 | <0.001 |
| WEEK 24 | | PLA | 31 | −7.2 | | 165 | −1.4 | | |
| | | LS Mean Diff. | 60 | 12.4 | | 334 | 1.7 | | |
| | ADCS-ADL | MEM | 29 | −2.1 | 0.0238 | 91 | −1.8 | 0.1903 | 0.028 |
| | (change) | PLA | 32 | −5.7 | | 95 | −2.7 | | |
| | | LS Mean Diff. | 61 | 3.6 | | 186 | 0.9 | | |
| | CIBIC-PLUS | MEM | 29 | 4.17 | 0.0020 | 91 | 4.45 | 0.4870 | 0.027 |
| | | PLA | 31 | 5.35 | | 95 | 4.53 | | |
| | | Raw Mean Diff. | 60 | −1.18 | | 186 | −0.08 | | |

TABLE 16
Antipsychotics usage adjusted analysis at last week (ITT, LOCF)

| | | | ANTIPSYCHOTICS | | | ANTIPSYCHOTICS | | | |
|---|---|---|---|---|---|---|---|---|---|
| | | TREATMENT | USAGE “YES” | | | USAGE “YES” | | | OVERALL MEM v. PLACEBO |
| STUDY | PARAMETER | GROUP | N = y | mean | p-value | N = n | mean | p-value | p-value |
| MD-01 | SIB (change) | MEM | 38 | −6.2 | 0.4149 | 132 | −0.5 | 0.1685 | 0.616 |
| WEEK 24 | | PLA | 33 | −4.3 | | 132 | −2.2 | | |
| | | LS Mean Diff. | 71 | −1.9 | | 264 | 1.7 | | |
| | ADCS-ADL | MEM | 38 | −3.8 | 0.9173 | 133 | −0.8 | 0.2806 | 0.282 |
| | (change) | PLA | 33 | −3.8 | | 132 | −1.7 | | |
| | | LS Mean Diff. | 71 | −0.15 | | 265 | 0.8 | | |
| | CIBIC-PLUS | MEM | 38 | 4.63 | 0.9924 | 133 | 4.26 | 0.0699 | 0.182 |
| | | PLA | 33 | 4.73 | | 130 | 4.52 | | |
| | | Raw Mean Diff. | 71 | −0.1 | | 263 | −0.26 | | |

TABLE 17
Antipsychotics usage adjusted analysis at last week (ITT, LOCF)

| | | | ANTIPSYCHOTICS | | | ANTIPSYCHOTICS | | | |
|---|---|---|---|---|---|---|---|---|---|
| | | TREATMENT | USAGE “YES” | | | USAGE “YES” | | | OVERALL MEM v. PLACEBO |
| STUDY | PARAMETER | GROUP | N = y | mean | p-value | N = n | mean | p-value | p-value |
| MZ-9605 | SIB (change) | MEM | 13 | −2.5 | 0.0144 | 113 | −4.1 | 0.0029 | 0.0003 |
| WEEK 28 | | PLA | 23 | −13.0 | | 103 | −9.1 | | |
| | | LS Mean Diff. | 36 | 10.6 | | 216 | 5.1 | | |
| | ADCS-ADL | MEM | 13 | −5.5 | 0.6763 | 113 | −2.7 | 0.0299 | 0.0217 |
| | (change) | PLA | 23 | −6.7 | | 103 | −4.7 | | |
| | | LS Mean Diff. | 36 | 0.9 | | 216 | 1.9 | | |
| | CIBIC-PLUS | MEM | 13 | 4.62 | 0.5550 | 113 | 4.47 | 0.1000 | 0.0644 |
| | | PLA | 23 | 4.91 | | 103 | 4.69 | | |
| | | Raw Mean Diff. | 36 | −0.29 | | 216 | −0.22 | | |

TABLE 18
Antipsychotics usage adjusted analysis at last week (ITT, LOCF)

| | | | ANTIPSYCHOTICS | | | ANTIPSYCHOTICS | | | |
|---|---|---|---|---|---|---|---|---|---|
| | | TREATMENT | USAGE “YES” | | | USAGE “YES” | | | OVERALL MEM v. PLACEBO |
| STUDY | PARAMETER | GROUP | N = y | mean | p-value | N = n | mean | p-value | p-value |
| MD-12 | ADAS-cog | MEM | 23 | 3.2 | 0.8117 | 189 | 0.1 | 0.2261 | 0.184 |
| WEEK 24 | (change) | PLA | 28 | 2.8 | | 184 | 0.8 | | |
| | | LS Mean Diff. | 51 | .4 | | 373 | −0.74 | | |
| | ADAS-COG | MEM | 23 | −6.0 | 0.8429 | 191 | −2.5 | 0.7635 | 0.816 |
| | (change) | PLA | 28 | −6.4 | | 185 | −2.3 | | |
| | | LS Mean Diff. | 51 | .43 | | 376 | −0.24 | | |
| | CIBIC-PLUS | MEM | 23 | 4.74 | 0.4860 | 191 | 4.33 | 0.5690 | 0.843 |
| | | PLA | 28 | 4.64 | | 185 | 4.39 | | |
| | | Raw Mean Diff. | 51 | 0.1 | | 376 | −0.06 | | |

TABLE 19
Antipsychotics usage adjusted analysis at last week (ITT, LOCF)

| | | | |
|---|---|---|---|
| | ANTIPSYCHOTICS | ANTIPSYCHOTICS | |
| TREATMENT | USAGE “YES” | USAGE “YES” | OVERALL MEM v. PLACEBO |

| STUDY | PARAMETER | GROUP | N = y | mean | p-value | N = n | mean | p-value | p-value |
|---|---|---|---|---|---|---|---|---|---|
| MD-10 | ADAS-cog | MEM | 20 | 1.42 | 0.7026 | 175 | −0.54 | 0.0044 | 0.003 |
| WEEK 24 | (change) | PLA | 22 | 2.20 | | 176 | 1.38 | | |
| | | LS Mean Diff. | 42 | −0.74 | | 351 | −1.93 | | |
| | ADAS-COG | MEM | 20 | −7.95 | 0.9950 | 176 | −2.69 | 0.7387 | 0.890 |
| | (change) | PLA | 22 | −7.86 | | 176 | −3.05 | | |
| | | LS Mean Diff. | 42 | −0.017 | | 352 | 0.31 | | |
| | CIBIC-PLUS | MEM | 20 | 4.40 | 0.5262 | 176 | 4.18 | 0.0184 | 0.004 |
| | | PLA | 22 | 4.91 | | 175 | 4.47 | | |
| | | Raw Mean Diff. | 42 | −0.51 | | 351 | −0.29 | | |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

1. A method of treating a behavioral disorder in a subject in need thereof, comprising administering an effective amount of a 1-aminocyclohexane in a pharmaceutically acceptable carrier.

2. The method of claim 1, wherein the 1-aminocyclohexane is selected from memantine and neramexane, and pharmaceutically acceptable salts thereof.

3. The method of claim 2, wherein the 1-aminocyclohexane is memantine hydrochloride.

4. The method of claim 1, wherein the behavioral disorder is selected from the group consisting of delusions, hallucinations, agitation/aggression, depression/dysphoria, anxiety, elation/euphoria, apathy/indifference, disinhibition, irritability/lability, aberrant motor activity, nighttime behavior, and appetite/eating changes.

5. The method of claim 1, further comprising administering an acetylcholinesterase inhibitor or an antipsychotic.

6. The method of claim 5, wherein the acetylcholinesterase inhibitor is donepezil.

7. The method of claim 5, wherein the antipsychotic is an atypical antipsychotic.

8. A method for the treatment of agitation in a subject in need thereof, comprising administering an effective amount of a 1-aminocyclohexane in a pharmaceutically acceptable carrier.

9. The method of claim 8, wherein the 1-aminocyclohexane is selected from memantine and neramexane.

10. The method of claim 8, wherein the 1-aminocyclohexane is memantine hydrochloride.

11. The method of claim 10, wherein the memantine is administered in a dose of from about 5 to 60 mg/day.

12. The method of claim 11, wherein the dose of memantine is from about 10 to 40 mg/day.

13. The method of claim 8, wherein the agitation is associated with a disorder selected from the group consisting of depression, a mood disorder, substance abuse withdrawal, selective serotonin reuptake inhibitor withdrawal, traumatic brain injury, terminal illness, being in the intensive care unit, post-operative agitation, post-anesthetic agitation, and a pediatric disorder.

14. The method of claim 13, wherein the mood disorder is schizophrenia or bipolar disorder.

15. The method of claim 13, wherein the pediatric disorder is depression, attention deficit disorder (with and without hyperactivity), conduct disorder, oppositional defiant disorder, and separation anxiety disorder.

16. The method of claim 8, wherein the agitation is associated with Alzheimer's disease.

17. The method of claim 16, wherein the subject is concurrently treated with an antipsychotic.

18. The method of claim 16 wherein the subject has score of ≧4 on the Neuropsychiatric Inventory clinical scale.

19. The method of claim 16, wherein the memantine is administered in a dose of from about 5 to 60 mg/day.

20. The method of claim 19, wherein the dose of memantine is from about 10 to 40 mg/day.
